(19) United States
(12) Patent Application Publication
(10) Pub. No.: US 2009/0233008 A1
(43) Pub. Date: Sep. 17, 2009

(54) CELLULOSE ACYLATE GRAINS AND METHOD FOR PRODUCING THEM, CELLULOSE ACYLATE FILM AND METHOD FOR PRODUCING IT, POLARIZER, OPTICAL COMPENSATORY FILM, ANTIREFLECTION FILM AND LIQUID-CRYSTAL DISPLAY DEVICE

(75) Inventors: Satoshi Higuchi, Kanagawa (JP); Akihiro Matsufuji, Kanagawa (JP); Tetsuya Yoshida, Shizuoka (JP); Yasutomo Goto, Shizuoka (JP); Kiyokazu Hashimoto, Kanagawa (JP)

Correspondence Address: BUCHANAN, INGERSOLL & ROONEY PC, POST OFFICE BOX 1404, ALEXANDRIA, VA, 22313-1404, US

(73) Assignee: FujiFilm Corporation, Tokyo (JP)

(21) Appl. No.: 11/816,585

(22) PCT Filed: Dec. 21, 2006
(86) PCT No.: PCT/JP2006/326172

§ 371 Date: Aug. 17, 2007

(30) Foreign Application Priority Data
Dec. 21, 2005 (JP) 2005-367680
Jul. 28, 2006 (JP) 2006-207016

(51) Int. Cl. G02B 1/08; C07H 3/00; B29C 47/60; B29C 47/88; B29C 55/00; G02B 5/30

(52) U.S. Cl. 428/1.31; 536/63; 264/211.23; 264/211.12; 264/330; 264/291

(57) ABSTRACT

Cellulose acylate grains having a heat quantity of crystalline fusion of at most 10 J/g. A film prepared by melt-casting the grains is free from the problem of yellowing when built in liquid-crystal display devices.

### TECHNICAL FIELD

The present invention relates to cellulose acylate grains, a cellulose acylate film, and methods for producing them. The invention also relates to a polarizer, an optical compensatory film, an antireflection film and a liquid-crystal display device that comprise a cellulose acylate film having excellent optical properties.

### BACKGROUND ART

Heretofore, in producing cellulose acylate films for use in liquid-crystal image display devices, a solution-casting method has been principally carried out, which comprises dissolving cellulose acylate in a chlorine-containing organic solvent such as dichloromethane, casting it on a substrate, and drying it to form a film. Dichloromethane, a type of a chlorine-containing organic solvent has been favorably used as a solvent for cellulose acylate, since it is a good solvent for cellulose acylate and has a low boiling point (about 40° C.), therefore having the advantage of easy vaporization in a film formation step and in a drying step.

Recently, however, from the viewpoint of environmental protection, it has become strongly required to retard release of chlorine-containing organic solvent and other organic solvents. Accordingly, some countermeasures have heretofore been tried and employed for retarding the release of organic solvent as much as possible, for example, by using a more severely-controlled closed system enough to prevent the leakage of organic solvent from it, or by leading the organic solvent, if any, leaked out from a film-forming system into a gas absorption column to adsorb it before the organic solvent is released in outdoor air, or by burning the organic solvent with flames, or by decomposing it with electron beams. Even by these countermeasures, however, it is still impossible to completely prevent the release of organic solvent, and further improvements are required.

A method of film formation with no use of organic solvent has been developed (see JP-A-2000-352620), which is melt-casting film formation of cellulose acylate. This reference says that the carbon chain of the ester group in cellulose acylate is prolonged so as to lower the melting point of the polymer for easy melt-casting film formation of the polymer. Concretely, it describes changing cellulose acetate into cellulose propionate.

### DISCLOSURE OF THE INVENTION

We, the present inventors tried forming a polarizer, using a film produced according to the melt-casting film formation method described in Patent Reference 1, and tried building the polarizer in liquid-crystal display devices (LCD), but we have known that, when the devices are used for a long period of time, then they have problem of increased yellowing. For example, we have known that, when the liquid-crystal display devices are subjected to a forced aging test (for example, at 80° C. for 1000 hours) that corresponds to actual use thereof for a period of their durability, then they resulted in significant yellowing. The yellowing reduces the commercial value of the liquid-crystal display devices, and therefore, it is necessary to develop films that are free from the problem of yellowing.

In consideration of the above-mentioned prior-art problems, we, the present inventors have further studied for the purpose of providing films that do not yellow when built in liquid-crystal display devices and when used for a long period of time, and for the purpose of providing grains for forming the films.

We, the present inventors have assiduously analyzed the reasons of yellowing that may occur when liquid-crystal display devices are used for a long period of time, and, as a result, have found that the decomposition products produced in melt formation of films may undergo aged chemical change to cause yellowing. Based on this finding, we have succeeded in reducing the crystallization of cellulose acylate grains for use in melt-casting film formation and in solving the problem of yellowing. Accordingly, we have provided the present invention that comprises the following constitution.

[1] Cellulose acylate grains having a heat quantity of crystalline fusion of at most 10 J/g. Since the cellulose acylate grains of the invention have a low crystallinity, they can be melt in a kneading extruder for a short period of time. When the cellulose acylate grains of the invention is used in a melt-casting film formation process, the heat quantity required for melting the resins can be reduced whereby a thermal decomposition of the resins causing aged discoloration is prevented. The cellulose acylate film produced by using the cellulose acylate grains of the invention is, when built in a liquid-crystal display device and used for a long period of time, sufficiently prevented from yellowing.

[2] Cellulose acylate grains of [1], wherein the number of acicular impurities is at most 50/mg.

[3] Cellulose acylate grains of [1] or [2], having a sulfate group content of from 0 ppm to less than 200 ppm.

[4] Cellulose acylate grains of any one of [1] to [3], wherein the ratio of (sum of the molar amount of alkali metal and the molar amount of Group-2 metal)/(the molar amount of sulfate group) is from 0.3 to 3.0.

[5] Cellulose acylate grains of [4], wherein the Group-2 metal is calcium.

[6] Cellulose acylate grains of any one of [1] to [5], satisfying the following formulae (S-1) to (S-3):

2.6≦X+Y≦3.0, (S-1)

0≦X≦1.8, (S-2)

1.0≦Y≦3.0; (S-3)

wherein X means a degree of substitution of the hydroxyl group of cellulose for an acetyl group; Y means a total degree of substitution of the hydroxyl group of cellulose for a propionyl group, a butyryl group, a pentanoyl group and a hexanoyl group.

[7] Cellulose acylate grains of any one of [1] to [6], which are pellets.

[8] A method for producing cellulose acylate grains, which comprises kneading a cellulose acylate resin in a double-screw kneading extruder at a screw revolution of from 50 to 300 rpm and under a resin-kneading pressure of 2 to 9 MPa.

[9] The method for producing cellulose acylate grains of [8], which includes kneading and extruding the cellulose acylate resin at 160° C. to 220° C. and pelletizing it.

[10] The method for producing cellulose acylate grains of [8] or [9], wherein the resin is pelletized by controlling the inner pressure of the double-screw kneading extruder to lower than 1 atmospheric pressure.

[11] The method for producing cellulose acylate grains of any one of [8] to [10], wherein the resin is pelletized while an inert gas is introduced into the double-screw kneading extruder.

[12] The method for producing cellulose acylate grains of any one of [9] to [11], which includes grinding the pellets formed through pelletization.

[13] The method for producing cellulose acylate grains of any one of [8] to [12], which includes reacting the cellulose acylate with a carbonate, hydrogencarbonate, hydroxide or oxide of at least one metal selected from the group consisting of sodium, potassium, magnesium and calcium for neutralization before the kneading.

[14] A method for producing cellulose acylate grains, which comprises preparing a cellulose acylate solution by dissolving a cellulose acylate in a solvent having an SP value of from 7 to 10, and then solidifying the cellulose acylate.

[15] The method for producing cellulose acylate grains of [14], wherein the solvent having an SP value of from 7 to 10 is an ester solvent having an SP value of from 7 to 10, a halogenated hydrocarbon solvent having an SP value of from 7 to 10 or a ketone solvent having an SP value of from 7 to 10.

[16] The method for producing cellulose acylate grains of [14] or [15], wherein the solidification is attained by drying the cellulose acylate solution to remove the solvent.

[17] The method for producing cellulose acylate grains of [14] or [15], wherein the solidification is attained by introducing the cellulose acylate solution into a poor solvent to thereby precipitate the cellulose acylate.

[18] A method for producing a cellulose acylate film, which comprises melt-casting cellulose acylate grains of any one of [1] to [7] into a film.

[19] The method for producing a cellulose acylate film of [18], wherein the film is formed by the use of a touch roll under a linear pressure of from 3 kg/cm to 100 kg/cm.

[20] The method for producing a cellulose acylate film of [18], wherein the film is formed by the use of a touch roll under a contact pressure of from 0.3 MPa to 3 MPa.

[21] The method for producing a cellulose acylate film of any one of [18] to [20], which further includes stretching the formed cellulose acylate film in at least one direction by from 1% to 300%.

[22] A cellulose acylate film produced according to the production method of any one of [18] to [21].

[23] A cellulose acylate film formed of the cellulose acylate grains of any one of [1] to [7], which has a residual solvent content of at most 0.01% by mass.

[24] A polarizer comprising a polarizing film and at least one layer of the cellulose acylate film of [22] or [23] laminated thereon.

[25] An optical compensatory film comprising the cellulose acylate film of [22] or [23] as the substrate thereof.

[26] An antireflection film comprising the cellulose acylate film of [22] or [23] as the substrate thereof.

[27] A liquid-crystal display device comprising at least one of the polarizer of [24], the optical compensatory film of [25], and the antireflection film of [26].

The method for producing cellulose acylate grains of the invention may include activating cellulose, acylating the cellulose and/or cleaning with sulfuric acid prior to the neutralization.

The cellulose acylate film of the invention is, when built in a liquid-crystal display device and used for a long period of time, prevented from yellowing. Accordingly, the cellulose acylate film of the invention is extremely useful as polarizer, optical compensatory film and antireflection film. According to the production method for cellulose acylate grains of the invention, the cellulose acylate film may be produced in a simplified manner.

### BRIEF DESCRIPTION OF THE DRAWINGS

FIG. 1 is a schematic view showing the outline of one embodiment of an apparatus for carrying out melt-casting film formation according to the invention. In the drawing, 101 is a kneading extruder, 102 is a gear pump, 103 is a filter, 104 is a die, 105 is a touch roll, 106 is a casting chill drum, 107 is a cellulose acylate, 108 is a machine-direction stretching zone, 109 is a cross-direction stretching zone, 110 is a winding up zone.

### BEST MODE FOR CARRYING OUT THE INVENTION

The cellulose acylate grains, the cellulose acylate film, and their production methods and their applications are described in detail hereinunder.

In this description, the term “grains” is a concept that broadly includes any and every granular matter having a size of from 0.01 mm³ to 100000 mm³ or so. Also in this description, the term “pellets” is a concept within a range of “grains”, and this means a granular matter having a size of 1 mm³ to 500 mm³ or so.

The description of the constitutive elements of the invention given hereinunder may be for some typical embodiments of the invention, to which, however, the invention should not be limited. In this description, the numerical range expressed by the wording “a number to another number” means the range that falls between the former number indicating the lowermost limit of the range and the latter number indicating the uppermost limit thereof.

### Retardation of Yellowing:

In the invention, the cellulose acylate grains to be used for producing cellulose acylate films are characterized by the following, in order to prevent the formed films from yellowing when built in liquid-crystal display devices and when used for a long period of time. In addition, the method for producing cellulose acylate grains and the method for producing cellulose acylate films are also characterized by the following. These are described in order.

### (1) Characteristics of Cellulose Acylate Grains:

As in the above, in the invention, cellulose acylate grains having a heat quantity of crystalline fusion of at most 10 J/g are used for producing cellulose acylate films in order that the films are, when built in liquid-crystal display devices and when used for a long period of time, prevented from yellowing. The heat quantity of crystalline fusion of the cellulose acylate grains is more preferably from 0 J/g to 7 J/g, even more preferably from 0 J/g to 5 J/g. By using the cellulose acylate grains of the invention having a small heat quantity of crystalline fusion, the grains can be rapidly melt in the melt-casting film formation process. The thermal decomposition of the resins that is the main cause of the yellowing was found to be prevented remarkably compared to the prior processes.

In the invention, the heat of crystalline fusion is obtained from the sum total of the areas of the heat absorption peaks in DSC (differential scanning calorimeter). When the absorption peak is not detected, the heat of crystalline fusion is expressed as 0 (J/g).

Preferably, the number of acicular impurities in the cellulose acylate grains of the invention is at most 50/mg, more preferably from 0 to 40/mg, even more preferably from 0 to 30/mg. When acicular impurities exist in the grains, then they act as nuclei and promote crystallization in the grains around them. Accordingly, when they are desired to be melted in the process of melt-casting film formation, then they may be thermally decomposed to increase aged discoloration. In particular, the filter disposed in a melt-casting film formation apparatus (in general, it is disposed between a kneading extruder and a die) has a large dead space, and a resin melt may stay therein and thermally decomposed to cause aged discoloration. When the cellulose acylate grains having a smaller amount of acicular impurities as above are used, then the thermal decomposition may be more effectively prevented.

The reason of acicular impurities is the unreacted cellulose still having remained after acylation of cellulose. Accordingly, for the purpose of reducing the number of acicular impurities, preferably employed are a method of previously activating the starting cellulose for acylation, for example, by swelling it, in order that the acylating agent (carboxylic acid anhydride) used could fully penetrate into it, and/or a method of filtering the acylated cellulose through filter paper or filter cloth to remove acicular impurities from it, and then putting it in a poor solvent to precipitate cellulose acylate.

Preferably, the sulfate group content of the cellulose acylate grains of the invention is from 0 ppm to less than 200 ppm, more preferably from 10 ppm to 160 ppm, even more preferably from 20 ppm to 120 ppm. The residual sulfate group as referred to herein is a sulfate group that exists in the cellulose acylate in the form of a bound sulfuric acid, a non-bound sulfuric acid, a salt, an ester or a complex thereof; and the sulfate group content means the total content of those sulfate groups. The sulfate group in cellulose acylate would be because sulfuric acid serving as an acylation catalyst may bond to the hydroxyl group of cellulose to form a sulfate ester, or may be caught by cellulose acylate as its free sulfuric acid or its salt, ester or complex therein, and they could not be removed in a washing step but remain in the polymer. The cellulose acylate grains having a sulfate group content of less than 200 ppm may be prepared by suitably washing the cellulose acylate in the process of producing it. The residual sulfate group may esterify with the residual hydroxyl group of cellulose acylate, but owing to the strong hydrogen bonding thereof, the cellulose acylate grains may readily aggregate together to promote the crystallization thereof. Accordingly, when the grains are melted in a melt-casting film formation process, then they are thermally decomposed to increase aged discoloration. Therefore, it is desirable that the sulfate group content is controlled to be less than 200 ppm, whereby the formation of thermally-decomposed products that may be a cause of aged discoloration of cellulose acylate, may be more effectively prevented.

Preferably, the cellulose acylate grains of the invention contains an alkali metal and a Group-2 metal element. One or more of these may be in the polymer either singly or as combined. Preferably, these compounds are added during the process of producing cellulose acylate, more preferably during the washing step after cellulose acylate production. Preferred alkali metals and Group-2 metal elements are magnesium, calcium and strontium; more preferred are magnesium and calcium; and even more preferred is calcium. Preferably, the alkali metal and the Group-2 metal element are added as weakly-alkaline compounds thereof, for example, as metal carbonates, metal hydrogencarbonates, metal hydroxides, or metal oxides. More preferred are hydroxide compounds or weakly acid-salt compounds; and even more preferred are hydroxide compounds. Above all, especially preferred are magnesium or calcium carbonates, hydrogencarbonates, hydroxides and oxides; more preferred is calcium hydroxide.

Adding an alkali metal and/or a Group-2 metal is effective for neutralizing the above-mentioned sulfuric acid. As a result, the formation of thermal decomposition products, which is a cause of aged discoloration of cellulose acylate, may be more effectively prevented. In the cellulose acylate grains of the invention, the ratio of (sum of the molar amount of alkali metal and the molar amount of Group-2 metal)/(the molar amount of sulfate group) is preferably from 0.3 to 3.0, more preferably from 0.4 to 2.5, even more preferably from 0.5 to 2.0.

The weight-average degree of polymerization of the cellulose acylate in the cellulose acylate grains of the invention is preferably from 250 to 500, more preferably from 330 to 480, even more preferably from 350 to 450. When cellulose acylate having such a low degree of polymerization is used, then the melt viscosity thereof in melt-casting film formation may be small to facilitate the intended melt-casting film formation. As a result, it is unnecessary to increase the melting temperature in the film formation process and therefore the formation of thermal decomposition products, which is a cause of aged discoloration of cellulose acylate, may be more effectively prevented. In addition, the low-molecular-weight polymer may prevent crystal formation and is therefore effective for reducing the amount of crystals in the grains. The degree of polymerization may lower with the increase in the acylation temperature and with the prolongation of the reaction time, and therefore the cellulose acylate for use herein may have a controlled desired degree of polymerization by controlling the acylation temperature and time.

Preferably, the cellulose acylate grains of the invention have a degree of substitution satisfying the following formulae (S-1) to (S-3), more preferably satisfying the following formulae (S-4) to (S-6), even more preferably satisfying the following formulae (S-7) to (S-9). Having the degree of substitution (composition) as defined herein, the crystal formation in the grains may be retarded. Specifically, a propionyl group, a butyryl group, a pentanoyl group and a hexanoyl group that are bulkier than an acetyl group are made to exist in the cellulose acylate grains of the invention along with an acetyl group as combined with them, whereby the molecular regularity of the grains may be broken and the crystal formation may be prevented. The degree of substitution may be controlled by controlling the amount of the acylating agent (acid anhydride) to be added to the reaction system.

### (2-1) Production of Grains by Melting Process:

The cellulose acylate grains of the invention may be produced through a step of melting a cellulose acylate resin (melting process). In particular, when pellets are produced, the melting process is employed.

Concretely, the cellulose acylate grains of the invention may be produced by kneading a cellulose acylate resin in a double-screw kneading extruder at a screw revolution of from 50 to 300 rpm under a resin-kneading pressure of 2 to 9 MPa. The screw revolution is more preferably from 80 to 250 rpm, and even more preferably from 100 to 230 rpm. The resin-kneading pressure is more preferably from 2 to 8 MPa, and even more preferably 3 to 6 MPa.

When such an inner pressure is applied thereto, the cellulose acylate resin, or that is, the starting material for grains may be filled up in the double-screw extruder used. As a result, the resin may be more efficiently kneaded, and the crystals may be more sufficiently melted while preventing thermal decomposition. In general, such an inner pressure is not applied to the resin-melting system. However, if the pressure is not applied thereto, the double-screw extruder may have a space around the screw therein, in which the resin may be strongly sheared and may thereby readily undergo thermal decomposition. This may be a cause of aged discoloration after film formation. The pressure control may be attained by providing a pressure control valve at the outlet port of the double-screw kneading extruder for use herein. In general, a double-screw kneading extruder is used at a revolution of at most 40 rpm, but in the invention, the extruder is preferably used under the condition as above. Accordingly, the residence time in the double-screw extruder may be shortened and the thermal decomposition may be more effectively prevented. The increase in the shear force by the high revolution may promote the fusion of crystals.

The pelletization is effected preferably at a temperature of from 160° C. to 220° C. inside the double-screw kneading extruder, more preferably from 170° C. to 215° C., even more preferably from 180° C. to 210° C. In general, a film-forming resin is melted at a high temperature of 230° C. or higher, but in the invention, the starting cellulose acylate resin is melted preferably at such a low temperature. Within the screw revolution range and the inner pressure range as above, the resin crystals may be melted, and such a low temperature range is enough in the invention. As a result, thermal decomposition that causes aged discoloration may be more effectively prevented.

In the invention, the resin is pelletized while the inner pressure of the double-screw kneading extruder used is preferably kept lower than 1 atmospheric pressure, more preferably from 0 to 0.8 atmospheric pressure, even more preferably from 0.1 to 0.6 atmospheric pressure. The reduced pressure may be attained by degassing the double-screw kneading extruder via the vent or hopper provided in the kneading zone thereof, by the use of a vacuum pump.

As the case may be, an inert gas may be introduced into the double-screw kneading extruder so as to make the oxygen concentration in the extruder preferably from 0 to 18%, more preferably from 0.5 to 16%, even more preferably from 1 to 14% during the pelletization therein. In this case, rare gases or nitrogen may be used for the inert gas, which may be introduced into the double-screw kneading extruder via the vent or hopper provided in the kneading zone of the extruder.

The pressure reduction and the inert gas injection may be effected independently, or may be effected simultaneously as combined as one preferred embodiment of pelletization.

The cellulose acylate pellets thus prepared in the manner as above are suitable for film formation by the use of a single-screw extruder in the subsequent film-forming step. A single-screw extruder may attain a constant resin melt extrusion per unit time, and it may prevent film thickness fluctuation.

When ground, the cellulose acylate pellets produced in the manner as above may be cellulose acylate grains having a smaller grain size. The resulting grains may also be used for film formation.

### (2-2) Production of Grains by Dissolving Process:

The cellulose acylate grains of the invention may be produced through a process of dissolving a cellulose acylate resin (dissolving process). Concretely, a cellulose acylate resin is dissolved in a solvent having an SP value of from 7 to 10, and then solidified to produce cellulose acylate grains of the invention. The SP value is more preferably from 7.5 to 9.7, and even more preferably from 8.0 to 9.5. The definition and the determination of the SP value (solubility parameter) as referred to herein are described in detail in the section of measurement methods given hereinunder. The solvent having an SP value of from 7 to 10 is preferably an ester solvent having an SP value of from 7 to 10, a halogenated hydrocarbon solvent having an SP value of from 7 to 10 or a ketone solvent having an SP value of from 7 to 10. Examples of the preferred solvent include acetone, methyl ethyl ketone, diethyl ketone, ethyl acetate, butyl acetate and dichloromethane. Among then, more preferable are acetone, methyl ethyl ketone, ethyl acetate, butyl acetate and dichloromethane.

The concentration of the cellulose acylate solution to be obtained after dissolution is preferably from 1% by mass to 40% by mass, more preferably from 3% by mass to 35% by mass, even more preferably from 5% by mass to 30% by mass. The dissolution temperature is preferably from 10° C. to 50° C., more preferably from 15° C. to 40° C.

The solidification may be attained by drying the solution to evaporate the solvent (drying method), or by putting the solution into a poor solvent for precipitation (precipitation method). The poor solvent is preferably water, or a mixed solvent of water and lower alcohol (e.g., methanol, ethanol, propanol).

When cellulose acylate is dissolved in a solvent having the above-mentioned SP value, the solvent has a suitable affinity for cellulose acylate and therefore cellulose acylate is prevented from aggregating to form crystals. Accordingly, the heat quantity of crystalline fusion of the cellulose acylate grains produced may be controlled to fall within the defined range of the invention.

Preferably, the cellulose acylate grains of the invention have a size of from 1 mm³ to 100000 mm³, more preferably from 2 mm³ to 50000 mm³, even more preferably from 3 mm³ to 10000 mm³. When the drying method is employed, then the solidified cellulose acylate may be ground for size control. When the precipitation method is employed, the size of the droplets of the cellulose acylate solution to be put into a poor solvent may be controlled, or the cellulose acylate solution added to a poor solvent may be stirred at high speed, whereby the size of the droplets of the cellulose acylate solution may be reduced to control the grains to be produced.

The cellulose acylate grains thus obtained in the manner as above are suitable for film formation to be attained by the use of a double-screw extruder in the subsequent melt-casting film formation process. In a double-screw extruder, the resin may be melted while a high shear force is applied thereto. In this, therefore, formation of fish eyes to be caused by un-melted cellulose acylate may be prevented. Preferably, the double-screw kneading extruder is driven to knead and melt the resin therein, at a screw revolution of from 50 to 300 rpm under a resin-kneading pressure of at most 10 MPa. More preferably, the screw revolution is from 80 to 250 rpm, and the resin-kneading pressure is from 1 MPa to 9 MPa; even more preferably, the screw revolution is from 100 to 230 rpm.

When the combination of cellulose acylate pellets and a single-screw extruder mentioned above is compared with the combination of cellulose acylate grains and a double-screw extruder, then the former is preferred to the latter in point of its capability of film thickness control that is a more important factor for optical films.

### (3) Production of Cellulose Acylate Film by the Use of Touch Roll:

In the invention, a cellulose acylate film is prepared from the cellulose acylate grains produced by the melting process or the dissolving process.

It is desirable to use a touch roll in preparing the cellulose acylate film. The touch roll is a roll to be disposed in the film-forming system in such a manner that the resin melt (resin in melt) having passed through a die from a melt extruder could be sandwiched between a roll onto which the resin melt is to be cast, and this touch roll.

The touch roll of the type may comprise an elastic layer formed on a metal shaft, which is further covered with an outer jacket and in which a liquid medium layer is filled between the elastic layer and the outer jacket. The layer thickness of the outer jacket is preferably from 0.05 mm to 7.0 mm, more preferably from 0.2 mm to 5.0 mm. Preferably, the casting roll and the touch roll both have a mirror surface having an arithmetical mean height Ra of at most 100 nm, more preferably at most 50 nm, even more preferably at most 25 nm. Concretely, for example, those described in JP-A-11-314263, JP-A-2002-36332, JP-A-11-235747, JP-A-2004-216717, JP-A-2003-145609, WO97/28950 are usable herein.

To that effect, since the touch roll is filled with a fluid inside its thin outer jacket, it may be elastically deformed as depressed by the pressure applied thereto when kept in contact with a casting roll. Accordingly, since the touch roll and the casting roll are in face-to-face contact with each other, their pressure is dispersed and they may attain a low surface pressure. Therefore, no residual strain remains in the film sandwiched between them, and the surface roughness of the film may be therefore removed. Preferably, the linear pressure of the touch roll is from 3 kg/cm to 100 kg/cm, more preferably from 5 kg/cm to 80 kg/cm, even more preferably from 7 kg/cm to 60 kg/cm. The linear pressure as referred to herein means a value to be obtained by dividing the power given to the touch roll by the width of die orifice.

Preferably, the force to press the touch roll is defined by a contact pressure thereto. The contact pressure means a value to be obtained by dividing the force to press the touch roll by the area in which the touch roll and the casting roll are kept in contact with each other. In the invention, the contact pressure is preferably from 0.3 MPa to 3 MPa, more preferably from 0.5 MPa to 2.5 MPa, even more preferably from 0.7 MPa to 2.0 MPa.

The temperature of the touch roll and the casting roll is preferably from 60° C. to 160° C., more preferably from 70° C. to 150° C., even more preferably from 80° C. go 140° C. The temperature control within the range may be attained by making a conditioned liquid or vapor run inside the roll.

When the touch roll of the type is used, then the film is cooled from both the casting roll and the touch roll and therefore its aged discoloration may be more effectively prevented. In the absence of the touch roll, the melt from a die may be cooled by the casting roll only on its one surface, and therefore the cooling speed is low. Accordingly, thermal decomposition of the cellulose acylate may be promoted, therefore causing aged discoloration of the resulting film. Since the casting roll is all the time exposed to air on its upper surface, the time for which the film on the roll is exposed to high temperatures may be short, but the film on the roll may readily undergo thermal decomposition. In that condition, therefore, rapidly cooling the film with the touch roll is especially effective.

### Cellulose Acylate:

Cellulose acylate for use in the invention is described below.

To the method of producing cellulose acylate for use in the invention, applicable is the description in Hatsumei Kyokai Disclosure Bulletin (No. 2001-1745, published by the Hatsumei Kyokai on Mar. 15, 2001), pp. 7-12. The amount added as referred to herein is in terms of % by mass relative to cellulose acylate.

### Starting Material:

The starting cellulose material for producing cellulose acylate is preferably one derived from hardwood pulp, softwood pulp, cotton linter.

### Activation:

Prior to acylation, the starting cellulose material is preferably processed with an activator (for activation). The activator is preferably acetic acid, propionic acid, butyric acid, more preferably acetic acid. The amount of the activator to be added is preferably from 5% to 10000%, more preferably from 10% to 2000%, even more preferably from 30% to 1000%. The method for its addition may be selected from spraying, dropwise application or dipping. The activation time is preferably from 20 minutes to 72 hours, more preferably from 20 minutes to 12 hours. The activation time is preferably from 0° C. to 90° C., more preferably from 20° C. to 60° C. If desired, an acylation catalyst such as sulfuric acid may be used along with the activator in an amount of from 0.1% by mass to 10% by mass.

The activation may reduce the amount of the above-mentioned acicular impurities in cellulose. Specifically, when the activation temperature is higher and when the activation time is longer, then the amount of the acicular impurities may be reduced more.

### Acylation:

Preferably, cellulose is reacted with a carboxylic acid anhydride in the presence of a Broensted acid or a Lewis acid serving as a catalyst (see Dictionary of Physic and Chemistry, 5th Ed., 2000), thereby acylating the hydroxyl group of cellulose.

For controlling the temperature increase owing to the reaction heat in acylation, it is desirable that the acylating agent is previously cooled. The acylation temperature is preferably from −50° C. to 50° C., more preferably from −30° C. to 40° C., even more preferably from −20° C. to 35° C. The lowermost reaction temperature is preferably −50° C. or higher, more preferably −30° C. or higher, even more preferably −20° C. or higher. The acylation time is preferably from 0.5 hours to 24 hours, more preferably from 1 hour to 12 hours, even more preferably from 1.5 hours to 10 hours.

For obtaining a cellulose mixed-acylate, for example, employable is a method of reacting cellulose with two different types of carboxylic acid anhydrides both serving as an acylating agent, as their mixture or by successively adding them; or a method of using a mixed acid anhydride of two different types of carboxylic acids (e.g., mixed acetic/propionic acid anhydride); or a method of reacting a carboxylic acid with a different carboxylic acid anhydride (e.g., acetic acid and propionic acid anhydride) in a reaction system to form a mixed acid anhydride (e.g., mixed acetic/propionic acid anhydride) followed by further reacting it with cellulose; or a method of once producing a cellulose acylate having a degree of substitution of less than 3, and then further acylating it with an acid anhydride or an acid halide at its remaining hydroxyl group.

Regarding the production of a cellulose acylate having a large degree of 6-substitution, referred to is the description in JP-A-11-5851, JP-A-2002-212338 and JP-A-2002-338601.

### Acid Anhydride:

For the carboxylic acid anhydride, the carboxylic acid preferably has from 2 to 22 carbon atoms. More preferred are acetic anhydride, propionic anhydride, butyric anhydride. Preferably, the acid anhydride is added to cellulose in an amount of from 1.1 to 50 equivalents to the hydroxyl group of cellulose, more preferably from 1.2 to 30 equivalents, even more preferably from 1.5 to 10 equivalents.

### Catalyst:

The acylation catalyst is preferably a Broensted acid or a Lewis acid, more preferably sulfuric acid or perchloric acid; and its preferred amount to be added is from 0.1 to 30% by mass, more preferably from 1 to 15% by mass, even more preferably from 3 to 12% by mass.

### Solvent:

The acylation solvent is preferably a carboxylic acid, more preferably a carboxylic acid having from 2 to 7 carbon atoms, even more preferably acetic acid, propionic acid, butyric acid. These solvents may be mixed for use herein.

### Reaction Stopper:

After the acylation, a reaction stopper is preferably added to the system. The reaction stopper may be any one capable of decomposing the acid anhydride, including, for example, water, alcohol (having from 1 to 3 carbon atoms), carboxylic acid (e.g., acetic acid, propionic acid, butyric acid). Above all, especially preferred is a mixture of water and a carboxylic acid (acetic acid). Regarding the blend ratio of water to carboxylic acid in the mixture, the amount of water is preferably from 5% by mass to 80% by mass, more preferably from 10% by mass to 60% by mass, even more preferably from 15% by mass to 50% by mass.

### Neutralizing Agent:

After stopping the acylation, a neutralizing agent may be added to the system. Preferred examples of the neutralizing agent are ammonium, organic quaternary ammonium, alkali metal, Group-2 metal, Group-3 to 12 metal or Group-13 to 15 element carbonates, hydrogencarbonates, salts with organic acids, hydroxides or oxides. Especially preferred are sodium, potassium, magnesium or calcium carbonates, hydrogencarbonates, hydroxides or oxides.

### Partial Hydrolysis:

Thus obtained, the cellulose acylate may have an overall degree of substitution of nearly 3, but for the purpose of obtaining an ester having a desired degree of substitution, the acylate may be kept in the presence of a small amount of a catalyst (generally, the remaining acylation catalyst such as sulfuric acid) and water, at 20 to 90° C. for a few minutes to a few days so as to partially hydrolyze the ester bond thereof, thereby reducing the degree of acyl substitution of the cellulose acylate to a desired degree. After this, the remaining catalyst may be neutralized with the above-mentioned neutralizing agent to stop the partial hydrolysis.

### Filtration:

In any stage from the acylation to reprecipitation, the mixture may be filtered. Preferably, the system may be diluted with a suitable solvent prior to the filtration. Through the filtration, unreacted acicular impurities may be removed. When the acicular impurities are previously removed through the above-mentioned activation treatment, then the filtration may be effected more efficiently, and this embodiment is more preferred.

### Reprecipitation:

The cellulose acylate solution may be mixed with water of an aqueous solution of a carboxylic acid (e.g., acetic acid, propionic acid) for reprecipitation. The reprecipitation may be effected in a continuous or batchwise mode.

### Washing:

After reprecipitation, the acylate is preferably washed. Water or hot water may be used for the washing. The termination of the washing may be confirmed through determination of pH, ion concentration or electroconductivity or through elementary analysis.

### Addition of Alkali Metal, Group-2 Metal:

After washed, an alkali metal or Group-2 metal compound as mentioned above is preferably added to the cellulose acylate. The compound may be added, for example, as follows: The compound is dissolved or dispersed in a solvent such as water, and then sprayed over cellulose acylate; or cellulose acylate is dipped and stirred in the solution or dispersion and then taken out through filtration.

### Drying:

Preferably, the cellulose acylate is dried at 50 to 160° C. so that it may have a water content of at most 2% by mass.

### Cellulose Acylate:

The cellulose acylate produced according to the above-mentioned production method is a polymer in which a part or all of the 2-, 3- and 6-positioned hydroxyl groups of the glucose unit bonding to the β-1,4-glycoside structure of cellulose are esterified with an acyl group. In the cellulose acylate for use in the invention, the hydroxyl groups of cellulose may be partially or entirely substituted with two or more different types of acyl groups.

Preferably, the cellulose acylate of the invention satisfies the following formulae (S-1) to (S-3):

2.6≦X+Y≦3.0, (S-1)

0≦X≦1.8, (S-2)

1.0≦Y≦3.0; (S-3)

wherein X means a degree of substitution of the hydroxyl group of cellulose for an acetyl group; Y means a total degree of substitution of the hydroxyl group of cellulose for a propionyl group, a butyryl group, a pentanoyl group and a hexanoyl group. When all the 2-, 3- and 6-positioned hydroxyl groups of cellulose are substituted with an acyl group, then the degree of substitution is 3.

More preferably, the cellulose acylate satisfies the following formulae (S-4) to (S-6):

2.7≦X+Y≦3.0, (S-4)

0≦X≦1.2, (S-5)

1.5≦Y≦3. (S-6)

Even more preferably, the cellulose acylate satisfies the following formulae (S-7) to (S-9):

2.8≦X+Y≦3.0, (S-7)

0≦X≦0.8, (S-8)

2.0≦Y≦3. (S-9)

When X+Y is 2.6 or more, then the hydrophilicity of the cellulose acylate is low and the acylate may be more efficiently dried.

When X is 1.8 or less, then the hydrophilicity of the cellulose acylate is low and the acylate may be more efficiently dried.

When Y is 1.0 or more, then the hydrophobicity of the cellulose acylate is relatively high, and the acylate may be efficiently dried.

### Additive:

### Heat Stabilizer:

For further improving the heat stability of the cellulose acylate in the invention, it is especially effective to add a heat stabilizer thereto. In particular, it is desirable to add a heat stabilizer thereto for the purpose of keeping the thermal stability of the cellulose acylate during its melt-casting film formation at high temperatures. Above all, it is desirable to add at least one phenolic stabilizer having a molecular weight of at least 500 and at least one selected from phosphite stabilizers having a molecular weight of at least 500 and thioether stabilizers having a molecular weight of at least 500.

Any known phenolic stabilizer may be favorably used herein. One preferred type of phenolic stabilizer is a hindered phenol stabilizer. Especially preferably, the hindered phenol stabilizer for use herein has a substituent at a position adjacent to the hydroxyphenyl group therein, in which the substituent is more preferably a substituted or unsubstituted alkyl group having from 1 to 22 carbon atoms, even more preferably a methyl group, an ethyl group, a propyl group, an isopropyl group, a butyl group, an isobutyl group, a tert-butyl group, a pentyl group, an isopentyl group, a tert-pentyl group, a hexyl group, an octyl group, an isooctyl group, a 2-ethylhexyl group. Stabilizers having both a hydroxyphenyl group and a phosphite group in one molecule are also preferred for use herein.

These are commercially available, and are sold, for example, by the following manufacturers. Irganox 1076, Irganox 1010, Irganox 3113, Irganox 245, Irganox 1135, Irganox 1330, Irganox 259, Irganox 565, Irganox 1035, Irganox 1098, Irganox 1425WL are available from Ciba Speciality Chemicals. Adekastab AO-50, Adekastab AO-60, Adekastab AO-20, Adekastab AO-70, Adekastab AO-80 are available from Asahi Denka Kogyo. Sumilizer BP-76, Sumilizer BP-101, Sumilizer GA-80 are available from Sumitomo Chemical. Seenox 326M, Seenox 336Bare available from Sypro.

Phosphite stabilizers having a molecular weight of at least 500 are effective as antioxidant, including, for example, compounds described in [0023] to [0039] in JP-A-2004-182979, and compounds described in JP-A-51-70316, JP-A-10-306175, JP-A-57-78431, JP-A-54-157159 and JP-A-55-13765. Other stabilizers such as those described in detail in Hatsumei Kyokai Disclosure Bulletin (No. 2001-1745, published by the Hatsumei Kyokai on Mar. 15, 2001), pp. 17-22 are also usable herein, as selected from them. These are also commercially available. For example, Adekastab 1178, 2112, PEP-8, PEP-24G, PEP-36G, HP-10 are available from Asahi Denka Kogyo; and Sandostab P-EPQ is available from Clariant.

In the invention, any known thioether stabilizer may be used. For example, Sumilizer TPL, TPM, TPS, TDP are commercially available from Sumitomo Chemical; and Adekastab AO-412S is available from Asahi Denka Kogyo.

When these stabilizers are used herein, it is desirable that at least one phenolic stabilizer, and at least one selected from phosphite stabilizers and thioether stabilizers are added to cellulose acylate each in an amount of from 0.02 to 3% by mass of the acylate, more preferably from 0.05 to 1% by mass. The blend ratio of the phenolic stabilizer, and the phosphite stabilizer and/or the thioether stabilizer is not specifically defined, but is preferably from 1/10 to 10/1 (by mass), more preferably from 1/5 to 5/1 (by mass), even more preferably from 1/3 to 3/1 (by mass), still more preferably from 1/3 to 2/1 (by mass).

In the invention, it is recommendable to use a stabilizer having both a hydroxyphenyl group and a phosphite group in one molecule. Its examples are described in JP-A-10-273494. One example of its commercial products is Sumilizer GP (by Sumitomo Chemical).

Further, the long-chain aliphatic amines described in JP-A-61-63686, the steric-hindered amine group-having compounds described in JP-A-6-329830, the hindered piperidinyl stabilizers described in JP-A-7-90270, and the organic amines described in JP-A-7-278164 are also usable herein.

Preferred amine stabilizers for use in the invention are Asahi Denka's commercial products Adekastab LA-57, LA-52, LA-67, LA-62, LA-77, and Ciba Speciality Chemicals' commercial products Tinubin 765, 144. The proportion of the amine to the stabilizer for use herein may be generally from 0.01 to 25% by weight or so.

### UV Absorbent:

The cellulose acylate may contain an UV inhibitor. UV inhibitors are described, for example, in JP-A-60-235852, JP-A-3-199201, JP-A-5-1907073, JP-A-5-194789, JP-A-5-271471, JP-A-6-107854, JP-A-6-118233, JP-A-6-148430, JP-A-7-11056, JP-A-7-11055, JP-A-8-29619, JP-A-B-239509, JP-A-2000-204173. The amount of the UV inhibitor that to be added is preferably from 0.01 to 2% by mass of the resin melt being prepared herein, more preferably from 0.01 to 1.5% by mass.

Commercially-available UV absorbents such as those mentioned below are also usable herein. Commercially-available benzotriazole compounds are Tinubin P (by Ciba Speciality Chemicals), Tinubin 234 (by Ciba Speciality Chemicals, Tinubin 320 (by Ciba Speciality Chemicals), Tinubin 326 (by Ciba Speciality Chemicals), Tinubin 327 (by Ciba Speciality Chemicals), Tinubin 328 (by Ciba Speciality Chemicals), Sumisorb 340 (by Sumitomo Chemical), Adekastab LA-31 (by Asahi Denka Kogyo). Commercially-available benzophenone-type UV absorbents are Seesorb 100 (by Sypro Chemical), Seesorb 101 (by Sypro Chemical), Seesorb 101S (by Sypro Chemical), Seesorb 102 (by Sypro Chemical), Seesorb 103 (by Sypro Chemical), Adekastab LA-51 (by Asahi Denka Kogyo), Chemisorb 111 (by Chemipro Chemical), Uvinul D-49 (by BASF). Commercially-available oxalic acid anilide-type UV absorbents are Tinubin 312 (by Ciba Speciality Chemicals), Tinubin 315 (by Ciba Speciality Chemicals). Commercially-available salicylic acid-type UV absorbents are Seesorb 201 (by Sypro Chemical), Seesorb 202 (by Sypro Chemical); and commercially-available cyanoacrylate-type UV absorbents are Seesorb 501 (by Sypro chemical), Uvinul N-539 (by BASF).

### Fine Particles:

Fine particles may be added to the cellulose acylate in the invention. Fine particles include those of an inorganic compounds and those of an organic compound, any of which may be used in the invention. Preferably, the fine particles to be in the cellulose acylate in the invention have a mean primary particle size of from 5 nm to 3 μm, more preferably from 5 nm to 2.5 μm, even more preferably from 20 nm to 2.0 μm. The amount of the fine particles to be added to the cellulose acylate may be from 0.005 to 1.0% by mass of the acylate, more preferably from 0.01 to 0.8% by mass, even more preferably from 0.02 to 0.4% by mass.

The inorganic compound includes SiO₂, ZnO, TiO₂, SnO₂, Al₂O₃, ZrO₂, In₂O₃, MgO, BaO, MoO₂, V₂O₅, talc, calcined kaolin, calcined calcium silicate, calcium silicate hydrate, aluminium silicate, magnesium silicate, and calcium phosphate. Preferred is at least one of SiO₂, ZnO, TiO₂, SnO₂, Al₂O₃, ZrO₂, In₂O₃, MgO, BaO, MoO₂ and V₂O₅; and more preferred are SiO₂, TiO₂, SnO₂, Al₂O₃, ZrO₂.

As fine particles of SiO₂, herein usable are commercial products of, for example, Aerosil R972, R972V, R974, R812, 200, 200V, 300, R202, OX50, TT600 (all by Nippon Aerosil). As fine particles of ZrO₂, usable are commercial products of, for example, Aerosil R976 and R811 (both by Nippon Aerosil) In addition, Seahostar KE-E10, E30, E40, E50, E70, E150, W10, W30, W50, P10, P30, P50, P100, P150, P250 (all by Nippon Shokubai) are also usable herein. Further Silica Microbeads P-400, 700 (by Shokubai Kasei Kogyo) are also usable. SO-G1, SO-G2, SO-G3, SO-G4, SO-G5, SO-G6, SO-E1, SO-E2, SO-E3, SO-E4, SO-E5, SO-E6, SO-C1, SO-C2, SO-C3, SO-C4, SO-C5, SO-C6 (all by Admatechs) are also usable. Further, Moritex's Silica Particles (produced by powdering aqueous dispersion) 8050, 8070, 8100, 8150 are also usable.

As the fine particles of an organic compound usable in the invention, preferred are polymers such as silicone resin, fluorine resin and acrylic resin; and more preferred is silicone resin. The silicone resin preferably has a three-dimensional network structure. Commercial products are usable herein, such as Tospearl 103, 105, 108, 120, 145, 3120 and 240 (all by Toshiba Silicone).

Preferably, the fine particles of an inorganic compound for use herein are subjected to surface treatment so that they may stably exist in the cellulose acylate film. It is also desirable that the inorganic fine particles are used herein after subjected to surface treatment. The surface treatment includes chemical surface treatment with a coupling agent, and physical surface treatment such as plasma discharge treatment or corona discharge treatment. In the invention, preferred is the surface treatment with a coupling agent. The coupling agent is preferably an organoalkoxy-metal compound (e.g., silane coupling agent, titanium coupling agent). For inorganic fine particles (especially SiO₂ particles) that may be used herein as fine particles, treatment with a silane coupling agent may be especially effective. Not specifically defined, the amount of the coupling agent may be preferably from 0.005 to 5% by mass, more preferably from 0.01 to 3% by mass of the inorganic fine particles.

### Plasticizer:

When a plasticizer is added to the cellulose acylate, then the crystalline melting temperature (Tm) of the acylate may be lowered. The plasticizer for use in the invention is not specifically defined in point of its molecular weight, but preferably has a high molecular weight. For example, its molecular weight is preferably at least 500, more preferably at least 550, even more preferably at least 600. Regarding its type, the plasticizer usable herein includes phosphates, alkylphthalylalkyl glycolates, carboxylates, fatty acid esters of polyalcohols. Regarding its morphology, the plasticizer may be solid or oily. Accordingly, the plasticizer is not specifically defined in point of its melting point or boiling point. In melt-casting film formation, a non-volatile plasticizer is especially preferred.

Even though a plasticizer having a high molecular weight is used, it may vaporize in a minor amount during film formation for a long period of time, and may deposit on a casting roll, and its deposit may be transferred onto the surface of the formed film, therefore causing surface defects of the film. Accordingly, use of no plasticizer is most preferred. For this, a cellulose acylate alone having a sufficiently low melt viscosity may be used. Concretely, cellulose acylate alone having a melt viscosity of at most 2000 Pa·s, more preferably at most 1500 Pa·s at its melting temperature (230° C.) may be used. The cellulose acylate of the type may be obtained by controlling the composition and the degree of polymerization of cellulose acylate as in the above.

### Lubricant:

A lubricant may be added to the cellulose acylate in the invention. The lubricant is preferably a fluorine-containing compound. The fluorine-containing compound may be a low-molecular compound or a polymer compound capable of expressing an effect of lubricant. The polymers described in JP-A-2001-269564 may be used as the polymer lubricant. As the fluorine-containing polymer lubricant, preferred are polymers produced through polymerization of a fluoroalkyl group-containing ethylenic unsaturated monomer as an indispensable ingredient. The fluoroalkyl group-containing ethylenic unsaturated monomer for the polymer may be any compound having an ethylenic unsaturated group and a fluoroalkyl group in the molecule, not specifically defined. Fluorine-containing surfactants are also usable herein, and nonionic surfactants are especially preferred.

### Production of Cellulose Acylate Film:

A method for producing a cellulose acylate film from a cellulose acylate is described below. In the invention, the film is preferably produced according to a melt-casting film formation method in which a mixture of cellulose acylate and additive is melted and formed into a film. When a residual solvent exists in the formed film, then its crystallization may go on while the film is dried with the result that the impact strength of the film may be lowered. Accordingly, in the invention, it is desirable that the residual solvent amount in the formed film is at most 0.01% by weight, more preferably 0%. In a melt-casting film formation method not using a solvent, the residual solvent amount may be 0%.

### Granulation:

In the above-mentioned method, cellulose acylate is granulated. The granulation may be attained by mixing cellulose acylate and additive and dissolving the mixture in a solvent, and then solidifying the solution by a drying method or a precipitation method, and optionally grinding the solid. Pelletization, if desired, may be attained as follows: Cellulose acylate and additive are mixed and dried in the manner as above, then melted in a double-screw kneading extruder and extruded out as strands, and they are cooled and solidified in water and pelletized into pellets. The resulting pellets may be ground into smaller grains.

### Drying:

Prior to the melt-casting film formation, it is desirable that the grains are dried so as to have a water content of at most 0.1% by mass, more preferably at most 0.01% by mass.

For this, the drying temperature is preferably from 40 to 180° C.; and the drying air rate is preferably from 20 to 400 m³/hr, more preferably from 100 to 250 m³/hr. The dew point of the drying air is preferably from 0 to −60° C., more preferably from −20 to −40° C.

### Melt Extrusion:

The dried cellulose acylate resin (grains such as pellets) is fed into the cylinder of a kneading extruder via the feed port thereof. The cellulose acylate grains may be used either alone or as mixed with any others. A single-screw extruder is more preferred when resin pellets are processed; but a double-screw extruder is more preferred when resin grains prepared by a dissolution method are processed. When a resin mixture is processed, any of single-screw or double-screw extruder may be used.

The screw compression ratio of the kneading extruder is preferably from 2.5 to 4.5, more preferably from 3.0 to 4.0. L (screw length)/D (screw diameter) is preferably from 20 to 70, more preferably from 24 to 50. The extrusion temperature is preferably from 190 to 240° C. Preferably, the barrel of the extruder in which the resin is melted is heated by a heater unit divided into 3 to 20 sections.

Preferably, the melting temperature is from 150° C. to 250° C., more preferably from 160° C. to 240° C., even more preferably from 170° C. to 235° C. In this case, it is desirable that the temperature on the inlet side (hopper side) is made lower and the temperature on the outlet side is made higher by from 10° C. to 60° C.

The screw may be a fullflight screw, a maddock screw, or a dalmage screw.

For preventing resin oxidation, it is more desirable that the inner atmosphere of the kneading extruder is an inert gas (e.g., nitrogen), or an extruder with a vent is used and it is degassed to be in vacuum.

### Filtration:

At the outlet port of the kneading extruder, the resin is preferably filtered through a breaker plate filter.

For precision filtration, it is desirable that a leaf-type disc filter unit is provided after the gear pump. The filtration may be effected in one stage or in multiple stages. Preferably, the filtration gauge is from 3 μm to 15 μm, more preferably from 3 μm to 10 μm. Preferably, the filter material is stainless steel or ordinary steel, more preferably stainless steel. The filter may be a knitted structure or a metal sintered structure, but the latter is preferred.

### Gear Pump:

For the purpose of improving the thickness accuracy (by reducing the resin jet fluctuation), a gear pump is preferably disposed between the kneading extruder and the die.

Accordingly, the resin pressure fluctuation at the die may be within ±1%.

For improving the constant feeding performance by the gear pump, it is also desirable to change the screw revolution so as to control the pressure before the gear pump to be constant. A high-precision gear pump comprising 3 or more gears is also effective. Since the residual matter in the gear pump may cause resin deterioration, the gear pump is preferably so designed that the amount of the residual matter therein may be as small as possible.

The temperature change at the adaptor that connects the kneading extruder and the gear pump, and the gear pump and the die is preferably as small as possible for the purpose of stabilizing the extrusion pressure. For this, an aluminium-buried heater is preferably used.

### Die:

So far as it is so designed that little resin melt may stay therein, any ordinary type of die, such as T-die, fishtail die or hanger coat die may be used herein. Just before the T-die, a static mixer may be disposed with no problem for the purpose of improving the uniformity of the resin temperature. In general, the clearance at the T-die outlet port is preferably from 1.0 to 5.0 times the film thickness, more preferably from 1.3 to 2 times.

Preferably, the die clearance is controllable to a distance of from 40 to 50 mm, more preferably to a distance of at most 25 mm. For reducing the film thickness fluctuation during film formation, it is also effective to measure the film thickness at the downstream site of the system and to feed back the found data for the die thickness control.

For providing functional layers as the outer layers, a multi-layer film formation apparatus may be used for producing a film having a two or more multi-layered structure.

The residence time taken by the resin that has entered the kneading extruder through its feeding port and goes out of it through its die may be from 2 minutes to 60 minutes, preferably from 4 minutes to 30 minutes.

### Casting:

The resin melt extruded out as a sheet through the die is cooled and solidified on a casting drum to form a film thereon. In this stage, preferably employed is an electrostatic charging method, an air knife method, an air chamber method, a vacuum nozzle method or a touch roll method for enhancing the airtight contact between the film and the drum. Also preferred is an edge pinning method (in which only both edges of the film are kept in airtight contact with the drum). Above all, especially preferred is a touch roll method.

Preferably, from 1 to 8 casting drums, more preferably from 2 to 5 casting drums are used for gradually cooling the film. Preferably, the roll diameter is from 50 mm to 5000 mm, more preferably from 150 mm to 1000 mm. The distance between these plural rolls is preferably from 0.3 mm to 300 mm as the surface-to-surface distance therebetween, more preferably from 3 mm to 30 mm. The temperature of the casting drum is preferably from 60° C. to 160° C., more preferably from 80° C. to 140° C.

Next, the film is peeled from the casting drum, then led through nip rolls and thereafter wound up. Thus obtained, the thickness of the unstretched film is preferably from 30 μm to 300 μm, more preferably from 40 μm to 200 μm, even more preferably from 50 μm to 150 μm.

### Winding:

Preferably, the film is trimmed at both edges thereof before wound up. The trimmed scraps may be recycled for the starting material for film. As the trimming cutter, usable is any of rotary cutter, shear blade, or knife. Its material may be any of carbon steel, stainless steel, or ceramics.

Preferably, the tension in winding up the film is from 1 kg/m-width to 50 kg/m-width, more preferably from 3 kg/m-width to 20 kg/m-width. Regarding the winding tension, the film may be wound up at a constant tension, but is preferably wound up as tapered according to the winding roll diameter.

It is necessary to control the draw ratio of the film between nip rolls so that the film does not receive any over tension than a defined level in the winding line.

Before wound up, the film may be laminated with any other film on at least one surface thereof.

Preferably, the width of the wound film is from 1 m to 3 m, more preferably from 1.2 m to 2.5 m. Preferably, the length of the wound film is from 1000 m to 8000 m, more preferably from 1500 m to 7000 m, even more preferably from 2000 m to 6000 m.

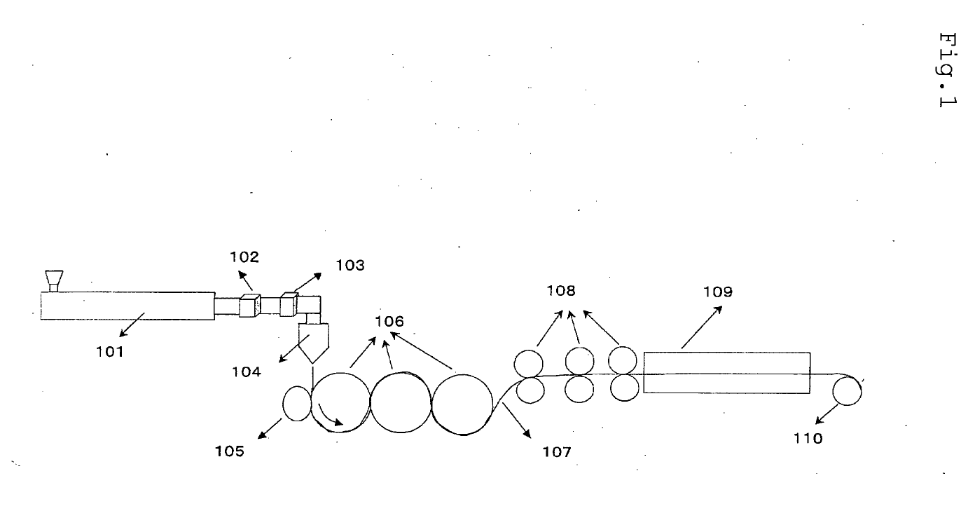

FIG. 1 is a schematic view showing the outline of an apparatus for melt-casting film formation preferably employable in the invention. In the drawing, 101 is a kneading extruder, 102 is a gear pump, 103 is a filter, 104 is a die, 105 is a touch roll, 106 is a casting chill drum, 107 is a cellulose acylate, 108 is a machine-direction stretching zone, 109 is a cross-direction stretching zone, 110 is a winding up zone. The stretching is described hereinunder. For unstretched film formation, the film being formed is, after left from the casting chill drums 106, directly wound up not being led through the stretching zone 108, 109.

### Physical Properties of Unstretched Cellulose Acylate Film:

Thus obtained, the unstretched cellulose acylate film preferably has Re=0 to 20 nm and Rth=0 to 80 nm, more preferably Re=0 to 10 nm and Rth=0 to 60 nm. Re and Rth indicate the in-plane retardation and the thickness-direction retardation, respectively, of the film. Re may be determined by applying light to the film in the normal direction of the film, using KOBRA 21ADH (by Oji Scientific Instruments). Rth is determined as follows: Based on the retardation data determined in three different directions, or that is, Re as above, and retardation values measured by applying light to the film in the direction tilted by +40° or −40° relative to the normal direction of the film with the slow axis as the tilt axis (rotation axis) thereof, Rth is computed. Preferably, the angle θ between the film-traveling direction (machine direction) and the slow axis of Re of the film is nearer to 0° or +90° or −90°.

Preferably, the whole light transmittance of the unstretched cellulose acylate film is from 90% to 100%. The haze of the film is generally from 0 to 1%, preferably from 0 to 0.6%.

Preferably, the thickness unevenness of the film is from 0% to 3% both in the machine direction and in the cross direction, more preferably from 0% to 2%.

Preferably, the tensile modulus of the film is from 1.5 kN/mm² to 3.5 kN/mm², more preferably from 1.8 kN/mm² to 2.6 kN/mm². The elongation at break of the film is preferably from 3% to 300%.

Tg of the film is preferably from 95° C. to 145° C. The thermal dimensional change of the film at 80° C. for 1 day is preferably from 0% to ±1%, more preferably from 0% to ±0.3% both in the machine direction and in the cross direction of the film.

The moisture permeability of the film at 40° C. and 90% RH is preferably from 300 g/m²·day to 1000 g/m²·day, more preferably from 500 g/m²·day to 800 g/m²·day. The equivalent water content of the film at 25° C. and 80% RH is preferably from 1% by mass to 4% by mass, more preferably from 1.5% by mass to 2.5% by mass.

### Stretching, and Physical Properties of Stretched Cellulose Acylate Film:

### Stretching:

The unstretched film may be stretched to control Re and Rth of the film.

The stretching temperature is preferably from Tg to (Tg+50° C.), more preferably from (Tg+5° C.) to (Tg+20° C.). Preferably, the draw ratio in stretching is from 1% to 300%, more preferably from 3% to 200% in at least one direction. More preferably, the draw ratio in one direction is made larger than that in the other direction; and the smaller draw ratio in one direction is preferably from 1% to 30%, more preferably from 3% to 20%, and the larger draw ratio in the other direction is preferably from 30% to 300%, more preferably from 40% to 150%. The stretching may be attained in one stage or in multiple stages. The draw ratio as referred to herein may be obtained according to the following formula:

Draw Ratio (%)=100×{(length after stretching)−(length before stretching)}/(length before stretching).

The stretching may be effected by the use of a nip roll or a tenter. A simultaneous biaxial stretching method as in JP-A-2000-37772, JP-A-2001-113591 and JP-A-2002-103445 may also be employed herein.

Re and Rth of the stretched cellulose acylate film preferably satisfy the following formulae:

Rth≧Re,

200≧Re≧0,

500≧Rth≧30.

More preferably, Re and Rth of the stretched cellulose acylate film satisfy the following formulae:

Rth≧Re×1.2,

100≧Re≧20,

350≧Rth≧80.

In machined-direction stretching, the angle θ formed by the film-traveling direction (machine direction) and the slow axis of Re of the film is preferably 0±3°, more preferably 0±1°. In cross-direction stretching, the angle is preferably 90±3° or −90±3°, more preferably 90±1° or −90±1°.

The thickness of the stretched cellulose acylate film is preferably from 15 μm to 200 μm, more preferably from 40 μm to 140 μm. The thickness unevenness of the film is preferably from 0% to 3%, more preferably from 0% to 1% both in the machine direction and in the cross direction thereof.

### Physical Properties:

The physical properties of the stretched cellulose acylate film preferably falls within the ranges mentioned below.

The tensile modulus of the film is preferably from 1.5 kN/mm² to 3.0 kN/mm², more preferably from 1.8 kN/mm² to 2.6 kN/mm².

The elongation at break of the film is preferably from 3% to 100%, more preferably from 8% to 50%.

Tg of the film is preferably from 95° C. to 145° C., more preferably from 105° C. to 135° C.

After left at 80° C. for 1 day, the thermal dimensional change of the film is preferably from 0% to ±1%, more preferably from 0% to ±0.3% both in the machine direction and in the cross direction thereof.

The moisture permeability at 40° C. and 90% RH of the film is preferably from 300 g/m²·day to 1000 g/m²·day, more preferably from 500 g/m²·day to 800 g/m²·day.

The equivalent water content of the film at 25° C. and 80% RH is preferably from 1% by mass to 4% by mass, more preferably from 1.5% by mass to 2.5% by mass.

The haze of the film is preferably from 0% to 3%, more preferably from 0% to 1%. The whole light transmittance of the film is preferably from 90% to 100%.

### Treatment of Cellulose Acylate Film:

The cellulose acylate film of the invention may be treated in various methods, and some preferred embodiments of its treatment are described below.

### Surface Treatment:

The cellulose acylate film may be optionally subjected to surface treatment to thereby improve the adhesiveness between the cellulose acylate film and various functional layers (e.g., undercoat layer, back layer) adjacent thereto. The surface treatment is, for example, glow discharge treatment, UV irradiation treatment, corona treatment, flame treatment, or acid or alkali treatment. The glow discharge treatment as referred to herein is preferably low-temperature plasma treatment to be effected under a low gas pressure of from 10⁻³ to 20 Torr, or plasma treatment under atmospheric pressure. The plasma-exciting vapor to be used in the plasma treatment is a vapor that is excited by plasma under the condition as above. The plasma-exciting vapor includes, for example, argon, helium, neon, krypton, xenon, nitrogen, carbon dioxide, flons such as tetrafluoromethane, and their mixtures. Their details are described in Hatsumei Kyokai Disclosure Bulletin (No. 2001-1745, published by the Hatsumei Kyokai on Mar. 15, 2001), pp. 30-32. For the plasma treatment under atmospheric pressure that has become specifically noted recently, preferably used is irradiation energy of from 20 to 500 KGy under 10 to 1000 Kev, more preferably from 20 to 300 KGy under 30 to 500 Kev. Of the above-mentioned treatments, more preferred is alkali saponification, and this is extremely effective for the surface treatment of cellulose acylate films.

For the alkali saponification, the film to be processed may be dipped in a saponification solution or may be coated with it. In the dipping method, the film may be led to pass through a tank of an aqueous NaOH or KOH solution having a pH of from 10 to 14 at 20 to 80° C., taking 0.1 minutes to 10 minutes, and then neutralized, washed with water and dried.

When the alkali saponification is attained according to a coating method, employable for it are a dip-coating method, a curtain-coating method, an extrusion-coating method, a bar-coating method and an E-type coating method. The solvent for the alkali saponification coating solution is preferably so selected that the saponification solution comprising it may well wet a transparent support to which the solution is applied, and that the solvent does not roughen the surface of the transparent support and may keep the support having a good surface condition. Concretely, alcohol solvents are preferred, and isopropyl alcohol is more preferred. An aqueous solution of surfactant may also be used as the solvent. The alkali to be in the alkali saponification coating solution is preferably an alkali soluble in the above-mentioned solvent. More preferably, it is KOH or NaOH. The pH of the saponification coating solution is preferably at least 10, more preferably at least 12. Regarding the reaction condition in alkali saponification, the reaction time is preferably from 1 second to 5 minutes at room temperature, more preferably from 5 seconds to 5 minutes, even more preferably from 20 seconds to 3 minutes. After the alkali saponification treatment, it is desirable that the saponification solution-coated surface of the film is washed with water or with an acid and then further washed with water. If desired, the coating saponification treatment may be effected continuously with the alignment film removal treatment that will be mentioned hereinunder. In that manner, the number of the processing steps in producing the film may be decreased. Concretely, for example, the saponification method is described in JP-A-2002-82226 and WO02/46809.

Preferably, the film of the invention is provided with an undercoat layer for improving the adhesiveness thereof to the functional layers to be formed thereon. The undercoat layer may be formed on the film after the above-mentioned surface treatment, or may be directly formed thereon with no surface treatment. The details of the undercoat layer are described in Hatsumei Kyokai Disclosure Bulletin (No. 2001-1745, published on Mar. 15, 2001 by the Hatsumei Kyokai), p. 32.

The step of surface treatment and undercoat layer formation may be carried out singly or as combined with the last step in the process of film formation. Further, the step may also be carried out along with the step of forming the functional groups to be mentioned hereinunder.

### Application of Cellulose Acylate Film of the Invention:

Preferably, the cellulose acylate film of the invention is combined with functional layers described in detail in Hatsumei Kyokai Disclosure Bulletin (No. 2001-1745, published on Mar. 15, 2001 by the Hatsumei Kyokai), pp. 32-45. Above all, it is desirable that the film is provided with a polarizing layer (for polarizer), an optically-compensatory layer (for optical compensatory sheet) and an antireflection layer (for antireflection film). These are described in order hereinunder.

### (1) Formation of Polarizing Layer (Construction of Polarizer)

### Materials:

At present, one general method of producing commercially-available polarizing films comprises dipping a stretched polymer in a solution containing iodine or dichroic dye in a bath to thereby infiltrate iodine or dichroic dye into the binder. As the polarizing film, a coated polarizing film such as typically that by Optiva Inc. may be utilized. Iodine and dichroic dye in the polarizing film are aligned in the binder and express the polarization property. The dichroic dye includes azo dyes, stilbene dyes, pyrazolone dyes, triphenylmethane dyes, quinoline dyes, oxazine dyes, thiazine dyes and anthraquinone dyes. Preferably, the dichroic dye is soluble in water. Also preferably, the dichroic dye has a hydrophilic substituent (e.g., sulfo, amino, hydroxyl). For example, the compounds described in Hatsumei Kyokai Disclosure Bulletin (No. 2001-1745, published on Mar. 15, 2001 by the Hatsumei Kyokai), p. 58 may be used as the dichroic dye herein.

For the binder for the polarizing film, usable are a polymer that is crosslinkable by itself, and a polymer that is crosslinkable with a crosslinking agent. These polymers may be combined for use herein. The binder includes, for example, methacrylate copolymers, styrene copolymers, polyolefins, polyvinyl alcohols, modified polyvinyl alcohols, poly(N-methylolacrylamides), polyesters, polyimides, vinyl acetate copolymers, carboxymethyl cellulose and polycarbonates, as in JP-A-8-338913, [0022]. In addition, a silane coupling agent may also be used as the polymer. Above all, water-soluble polymers (e.g., poly(N-methylolacrylamide), carboxymethyl cellulose, gelatin, polyvinyl alcohol, modified polyvinyl alcohol) are preferred; gelatin, polyvinyl alcohol and modified polyvinyl alcohol are more preferred; and polyvinyl alcohol and modified polyvinyl alcohol are most preferred. Especially preferably, two different types of polyvinyl alcohols or modified polyvinyl alcohols having a different degree of polymerization are combined for use herein. Preferably, the degree of saponification of polyvinyl alcohol for use herein is from 70 to 100%, more preferably from 80 to 100%. Also preferably, the degree of polymerization of polyvinyl alcohol is from 100 to 5000. Modified polyvinyl alcohols are described in JP-A-8-338913, JP-A-9-152509 and JP-A-9-316127. Two or more different types of polyvinyl alcohols and modified polyvinyl alcohols may be combined for use herein.

Preferably, the lowermost limit of the thickness of the binder is 10 μm. Regarding the uppermost limit of the thickness thereof, it is preferably thinner from the viewpoint of the light leakage resistance of liquid-crystal display devices comprising it. Concretely, for example, it is desirable that the thickness of the polarizing film is not larger than the same level as that of currently commercially-available polarizers (about 30 μm), more preferably it is at most 25 μm, even more preferably at most 20 μm.

The binder of the polarizing film may be crosslinked. A polymer or a monomer having a crosslinking functional group may be incorporated into the binder, or the binder polymer may be so designed that it has a crosslinking functional group by itself. The crosslinking may be attained through exposure to light or heat or through pH change, and it gives a binder having a crosslinked structure therein. The crosslinking agent is described in U.S. Reissue Pat. No. 23,297. A boron compound (e.g., boric acid, borax) may also be used as a crosslinking agent. The amount of the crosslinking agent to be added to the binder is preferably from 0.1 to 20% by mass of the binder. Within the range, the alignment of the polarizer element and the wet heat resistance of the polarizing film are both good.

After the crosslinking reaction, it is desirable that the amount of the unreacted crosslinking agent still remaining in the polarizing film is at most 1.0% by mass, more preferably at most 0.5% by mass. Within the range, the polarizing film may have good weather resistance.

### Stretching:

Preferably, the polarizing film is stretched (according to a stretching process) or rubbed (according to a rubbing process), and then dyed with iodine or dichroic dye.

In the stretching process, the draw ratio is preferably from 2.5 to 30.0 times, more preferably from 3.0 to 10.0 times. The stretching may be attained in dry in air. Contrary to this, the stretching may also be attained in wet while the film is dipped in water. Preferably, the draw ratio in dry stretching is from 2.5 to 5.0 times, and the draw ratio in wet stretching is from 3.0 to 10.0 times. The stretching may be effected once, or a few times. When the stretching is effected a few times, then the film may be more uniformly stretched even at a high draw ratio. The film may be stretched according to the following method.

Before stretched, PVA film is swollen. The degree of swelling of the film is from 1.2 to 2.0 times (in terms of the ratio by mass of the swollen film to the unswollen film). Next, the film is continuously conveyed via guide rolls, and led into a bath of an aqueous medium or into a dyeing bath of a dichroic substance solution. In the bath, in general, the film is stretched at a bath temperature of from 15 to 50° C., preferably from 17 to 40° C. The stretching may be effected by holding the film with two pairs of nip rolls, and the conveying speed of the latter-stage nip rolls is kept higher than that of the former-stage nip rolls. In view of the above-mentioned effects and advantages, the draw ratio in stretching (ratio of the length of stretched film/length of initial film—the same shall apply hereinunder) is preferably from 1.2 to 3.5 times, more preferably from 1.5 to 3.0 times. Next, the stretched film is dried at 50 to 90° C. to be a polarizing film.

### Lamination:

The saponified cellulose acylate film is laminated with a polarizing film prepared by stretching to thereby construct a polarizer. The direction in which the two are laminated is preferably so controlled that the casting axis direction of the cellulose acylate film crosses the stretching axis direction of the polarizer at an angle of 45 degrees.

Not specifically defined, the adhesive for the lamination may be an aqueous solution of a PVA resin (including modified PVA with any of acetoacetyl group, sulfonic acid group, carboxyl group and oxyalkylene group) or a boron compound. Above all, preferred are PVA resins. The thickness of the adhesive layer is preferably from 0.01 to 10 μm, more preferably from 0.05 to 5 μm, after dried.

The light transmittance of the thus-obtained polarizer is preferably higher, and the degree of polarization thereof is also preferably higher. Concretely, the transmittance of the polarizer preferably falls between 30 and 50% for the light having a wavelength of 550 nm, more preferably between 35 and 50%, most preferably between 40 and 50%. The degree of polarization of the polarizer preferably falls between 90 and 100% for the light having a wavelength of 550 nm, more preferably between 95 and 100%, most preferably between 99 and 100%.

Further, the thus-constructed polarizer may be laminated with a λ/4 plate to form a circularly-polarizing plate. In this case, the two are so laminated that the slow axis of the λ/4 plate meets the absorption axis of the polarizer at an angle of 45 degrees. In this, the λ/4 plate is not specifically defined but preferably has a wavelength dependency of such that its retardation is smaller at a lower wavelength. Further, it is also desirable to use a λ/4 plate that comprises a polarizing film of which the absorption axis is inclined by 20 to 70° relative to the machine direction and an optically-anisotropic layer of a liquid-crystalline compound.

### (2) Formation of Optical Compensatory Layer (Construction of Optical Compensatory Sheet):

An optically-anisotropic layer is for compensating the liquid-crystalline compound in a liquid-crystal cell at the time of black level of display in liquid-crystal display devices, and an optical compensatory sheet may be constructed by forming an alignment film on a cellulose acylate film followed by further forming thereon an optically-anisotropic layer.

### Alignment Film:

An alignment film is provided on the cellulose acylate film that has been processed for surface treatment as above. The film has the function of defining the alignment direction of liquid-crystal molecules. However, if a liquid-crystalline compound can be aligned and then its alignment state can be fixed as such, then the alignment film is not indispensable as a constitutive element, and may be therefore omitted as not always needed. In this case, only the optically-anisotropic layer on the alignment film of which the alignment state has been fixed may be transferred onto a polarizing element to construct the polarizer of the invention.

The alignment film may be formed, for example, through rubbing treatment of an organic compound (preferably polymer), oblique vapor deposition of an inorganic compound, formation of a microgrooved layer, or accumulation of an organic compound (e.g., ω-tricosanoic acid, dioctadecylmethylammonium chloride, methyl stearate) according to a Langmuir-Blodgett's method (LB film). Further, there are known other alignment films that may have an alignment function through impartation of an electric field or magnetic field thereto or through light irradiation thereto.

The alignment film is preferably formed through rubbing treatment of a polymer. In principle, the polymer to be used for the alignment film has a molecular structure that has the function of aligning liquid-crystalline molecules.

Preferably, the polymer for use in the invention has a crosslinking functional group (e.g., double bond)—having side branches bonded to the backbone chain thereof or has a crosslinking functional group having the function of aligning liquid-crystalline molecules introduced into the side branches thereof, in addition to having the function of aligning liquid-crystalline molecules.

The polymer to be used for the alignment film may be a polymer that is crosslinkable by itself or a polymer that is crosslinkable with a crosslinking agent, or may also be a combination of the two. Examples of the polymer are methacrylate copolymers, styrene copolymers, polyolefins, polyvinyl alcohols and modified polyvinyl alcohols, poly(N-methylolacrylamides), polyesters, polyimides, vinyl acetate copolymers, carboxymethyl cellulose and polycarbonates, as in JP-A-8-338913, [0022]. A silane coupling agent is also usable as the polymer. Preferably, the polymer is a water-soluble polymer (e.g., poly(N-methylolacrylamide), carboxymethyl cellulose, gelatin, polyvinyl alcohol, modified polyvinyl alcohol), more preferably gelatin, polyvinyl alcohol and modified polyvinyl alcohol, most preferably polyvinyl alcohol and modified polyvinyl alcohol. Especially preferably, two different types of polyvinyl alcohols or modified polyvinyl alcohols having a different degree of polymerization are combined for use as the polymer. Preferably, the degree of saponification of polyvinyl alcohol for use herein is from 70 to 100%, more preferably from 80 to 100%. Also preferably, the degree of polymerization of polyvinyl alcohol is from 100 to 5000.

The side branches having the function of aligning liquid-crystalline molecules generally have a hydrophobic group as the functional group. Concretely, the type of the functional group may be determined depending on the type of the liquid-crystalline molecules to be aligned and on the necessary alignment state of the molecules. For example, the modifying group of modified polyvinyl alcohol may be introduced into the polymer through copolymerization modification, chain transfer modification or block polymerization modification. Examples of the modifying group are a hydrophilic group (e.g., carboxylic acid group, sulfonic acid group, phosphonic acid group, amino group, ammonium group, amide group, thiol group), a hydrocarbon group having from 10 to 100 carbon atoms, a fluorine atom-substituted hydrocarbon group, a thioether group, a polymerizing group (e.g., unsaturated polymerizing group, epoxy group, aziridinyl group), and an alkoxysilyl group (e.g., trialkoxy group, dialkoxy group, monoalkoxy group). Specific examples of such modified polyvinyl alcohol compounds are described, for example, in JP-A-2000-155216, [0022] to [0145], and in JP-A-2002-62426, [0018] to [0022].

When crosslinking functional group-having side branches are bonded to the backbone chain of an alignment film polymer, or when a crosslinking functional group is introduced into the side chains of a polymer having the function of aligning liquid-crystalline molecules, then the polymer of the alignment film may be copolymerized with the polyfunctional monomer in an optically-anisotropic layer. As a result, not only between the polyfunctional monomers but also between the alignment film polymers, and even between the polyfunctional monomer and the alignment film polymer, they may be firmly bonded to each other in a mode of covalent bonding to each other. Accordingly, introducing such a crosslinking functional group into an alignment film polymer significantly improves the mechanical strength of the resulting optical compensatory sheet.

Preferably, the crosslinking functional group of the alignment film polymer contains a polymerizing group, like the polyfunctional monomer. Concretely, for example, those described in JP-A-2000-155216, [0080] to [0100] are referred to herein. Apart from the above-mentioned crosslinking functional group, the alignment film polymer may also be crosslinked with a crosslinking agent.

The crosslinking agent includes, for example, aldehydes, N-methylol compounds, dioxane derivatives, compounds capable of being active through activation of the carboxyl group thereof, active vinyl compounds, active halide compound, isoxazoles and dialdehyde starches. Two or more different types of crosslinking agents may be combined for use herein. Concretely, for example, the compounds described in JP-A-2002-62426, [0023] to [0024] are employable herein. Preferred are aldehydes of high reactivity, and more preferred is glutaraldehyde.

Preferably, the amount of the crosslinking agent to be added to polymer is from 0.1 to 20% by mass of the polymer, more preferably from 0.5 to 15% by mass. Also preferably, the amount of the unreacted crosslinking agent that may remain in the alignment film is at most 1.0% by mass, more preferably at most 0.5% by mass. When the crosslinking agent in the alignment film is controlled to that effect, then the film ensures good durability with no reticulation even though it is used in liquid-crystal display devices for a long period of time and even though it is left in a high-temperature high-humidity atmosphere for a long period of time.

Basically, the alignment film may be formed by applying the alignment film-forming material of the above-mentioned polymer to a crosslinking agent-containing transparent support, then heating and drying it (for crosslinking it) and then rubbing the thus-formed film. The crosslinking reaction may be effected in any stage after the film-forming material has been applied onto the transparent support, as so mentioned hereinabove. When a water-soluble polymer such as polyvinyl alcohol is used as the alignment film-forming material, then it is desirable that the solvent for the coating solution is a mixed solvent of a defoaming organic solvent (e.g., methanol) and water. The ratio by mass of water/methanol preferably falls between 0/100 and 99/1, more preferably between 0/100 and 91/9. The mixed solvent of the type is effective for preventing the formation of bubbles in the coating solution and, as a result, the surface defects of the alignment film and even the optically-anisotropic layer are greatly reduced.

For forming the alignment film, preferably employed is a spin-coating method, a dip-coating method, a curtain-coating method, an extrusion-coating method, a rod-coating method or a roll-coating method. Especially preferred is a rod-coating method. Also preferably, the thickness of the film is from 0.1 to 10 μm, after dried. The drying under heat may be effected at 20 to 110° C. For sufficient crosslinking, the heating temperature is preferably from 60 to 100° C., more preferably from 80 to 100° C. The drying time may be from 1 minute to 36 hours, but preferably from 1 to 30 minutes. The pH of the coating solution is preferably so defined that it is the best for the crosslinking agent used. For example, when glutaraldehyde is used, the pH of the coating solution is preferably from 4.5 to 5.5, more preferably 5.

The alignment film is provided on the transparent support or on the undercoat layer. The alignment film may be formed by crosslinking the polymer layer as above, and then rubbing the surface of the layer.

For the rubbing treatment, usable is any method widely employed for liquid crystal alignment treatment for LCD. Concretely, for example, the surface of the alignment film is rubbed in a predetermined direction by the use of paper, gauze, felt, rubber, nylon, or polyester fibers, whereby the film may be aligned in the intended direction. In general, a cloth uniformly planted with fibers having the same length and the same thickness is used, and the surface of the film is rubbed a few times with the cloth.

On an industrial scale, the operation may be attained by contacting a rolling rubbing roll to a polarizing layer-having film that is traveling in the system. Preferably, the circularity, the cylindricity, and the deflection (eccentricity) of the rubbing roll are all at most 30 μm each. Also preferably, the lapping angle of the film around the rubbing roll is from 0.1 to 90°. However, the film may be lapped at an angle of 360° or more for stable rubbing treatment, as in JP-A-8-160430. Preferably, the film traveling speed is from 1 to 100 m/min. The rubbing angle may fall between 0 and 60°, and it is desirable that a suitable rubbing angle is selected within the range. When the film is used in liquid-crystal display devices, the rubbing angle is preferably from 40 to 50°, more preferably 45°.

The thickness of the alignment film thus obtained is preferably from 0.1 to 10 μm.

Next, the liquid-crystalline molecules of the optically-anisotropic layer are aligned on the alignment film. Afterward, if desired, the polyfunctional monomers in the alignment film polymer and the optically-anisotropic layer are reacted, or the alignment film polymer is crosslinked with a crosslinking agent.

The liquid-crystalline molecules for use in the optically-anisotropic layer include rod-shaped liquid-crystalline molecules and discotic liquid-crystalline molecules. The rod-shaped liquid-crystalline molecules and the discotic liquid-crystalline molecules may be high-molecular liquid crystals or low-molecular liquid crystals. In addition, they include crosslinked low-molecular liquid crystals that do not exhibit liquid crystallinity.

### Rod-Shaped Liquid-Crystalline Molecules:

The rod-shaped liquid-crystalline molecules are preferably azomethines, azoxy compounds, cyanobiphenyls, cyanophenyl esters, benzoates, phenyl cyclohexanecarboxylates, cyanophenylcyclohexanes, cyano-substituted phenylpyrimidines, alkoxy-substituted phenylpyrimidines, phenyldioxanes, tolans and alkenylcyclohexylbenzonitriles.

The rod-shaped liquid-crystalline molecules include metal complexes. Liquid-crystal polymers that contain rod-shaped liquid-crystalline molecules in the repetitive units thereof are also usable herein as the rod-shaped liquid-crystalline molecules. In other words, the rod-shaped liquid-crystalline molecules for use herein may bond to a (liquid-crystal) polymer.

Rod-shaped liquid-crystalline molecules are described in Quarterly Journal of General Chemistry, Vol. 22, Liquid Crystal Chemistry (1994), Chaps. 4, 7 and 11, edited by the Chemical Society of Japan; Liquid Crystal Devices Handbook, edited by the 142nd Committee of the Nippon Academic Promotion, Chap. 3.

The birefringence of the rod-shaped liquid-crystalline molecule preferably falls between 0.001 and 0.7.

Preferably, the rod-shaped liquid-crystalline molecules have a polymerizing group for fixing their alignment state. The polymerizing group is preferably a radical-polymerizing unsaturated group or a cationic polymerizing group. Concretely, for example, there are mentioned the polymerizing groups and the polymerizing liquid-crystal compounds described in JP-A-2002-62427, [0064] to [0086].

### Discotic Liquid-Crystalline Molecules:

The discotic liquid-crystalline molecules include, for example, benzene derivatives as in C. Destrade et al's study report, Mol. Cryst., Vol. 71, p. 111 (1981); truxene derivatives as in C. Destrade et al's study report, Mol. Cryst., Vol. 122, p. 141 (1985), Physics Lett. A., Vol. 78, p. 82 (1990); cyclohexane derivatives as in B. Kohne et al's study report, Angew. Chem., Vol. 96, p. 70 (1984); and azacrown-type or phenylacetylene-type macrocycles as in J. M. Lehn et al's study report, J. Chem. Commun., p. 1794 (1985), J. Zhang et al's study report, J. Am. Chem. Soc., Vol. 116, p. 2655 (1994).

The discotic liquid-crystalline molecules include liquid-crystalline compounds in which the molecular center nucleus is radially substituted with side branches of a linear alkyl, alkoxy or substituted benzoyloxy group. Preferably, the molecules or the molecular aggregates of the compounds are rotary-symmetrical and may undergo certain alignment. It is not always necessary that, in the optically-anisotropic layer formed of such discotic liquid-crystalline molecules, the compounds that are finally in the optically-anisotropic layer are discotic liquid-crystalline molecules. For example, low-molecular discotic liquid-crystalline molecules may have a group capable of being reactive when exposed to heat or light, and as a result, they may polymerize or crosslink through thermal or optical reaction to give high-molecular compounds with no liquid crystallinity. Preferred examples of the discotic liquid-crystalline molecules are described in JP-A-8-50206. Polymerization of discotic liquid-crystalline molecules is described in JP-A-8-27284.

For fixing the discotic liquid-crystalline molecules through polymerization, the discotic core of the discotic liquid-crystalline molecules must be substituted with a polymerizing group. Preferably, the polymerizing group bonds to the discotic core via a linking group. Accordingly, the compounds of the type may keep their alignment state even after their polymerization. For example, there are mentioned the compounds described in JP-A-2000-155216, [0151] to [0168].

In hybrid alignment, the angle between the major axis (disc plane) of the discotic liquid-crystalline molecules and the plane of the polarizing film increases or decreases with the increase in the distance from the plane of the polarizing film in the depth direction of the optically-anisotropic layer. Preferably, the angle decreases with the increase in the distance. The angle change may be in any mode of continuous increase, continuous decrease, intermittent increase, intermittent decrease, change including continuous increase and continuous decrease, or intermittent change including increase and decrease. The intermittent change includes a region in which the tilt angle does not change in the midway of the thickness direction. The angle may include a region with no angle change so far as it increases or decreases as a whole. Preferably, the angle continuously varies.

The mean direction of the major axis of the discotic liquid-crystalline molecules on the polarizing film side may be controlled generally by suitably selecting the material of the discotic liquid-crystalline molecules or that of the alignment film or by suitably selecting the rubbing treatment method. The direction of the major axis of the discotic liquid-crystalline molecules (disc plane) on the surface side (on the external air side) may be controlled generally by suitably selecting the material of the discotic liquid-crystalline molecules or that of the additive to be used along with the discotic liquid-crystalline molecules. Examples of the additive that may be used along with the discotic liquid-crystalline molecules include, for example, plasticizer, surfactant, polymerizing monomer and polymer. Like in the above, the degree of the change of the major axis in the alignment direction may also be controlled by suitably selecting the liquid-crystalline molecules and the additive.

### Other Composition of Optically-Anisotropic Layer:

Along with the above-mentioned liquid-crystalline molecules, a plasticizer, a surfactant, a polymerizing monomer and others may be added to the optically-anisotropic layer for improving the uniformity of the coating film, the strength of the film and the alignment of the liquid-crystalline molecules in the film. Preferably, the additives have good compatibility with the liquid-crystalline molecules that constitute the layer and may have some influence on the tilt angle change of the liquid-crystalline molecules, not interfering with the alignment of the molecules.

The polymerizing monomer includes radical-polymerizing or cationic-polymerizing compounds. Preferred are polyfunctional radical-polymerizing monomers. Also preferred are those copolymerizable with the above-mentioned, polymerizing group-containing liquid-crystal compounds. For example, herein mentioned are the compounds described in JP-A-2002-296423, [0018] to [0020]. The amount of the compound to be added to the layer may be generally from 1 to 50% by mass of the discotic liquid-crystalline molecules in the layer, but preferably from 5 to 30% by mass.

The surfactant may be any known one, but is preferably a fluorine-containing compound. Concretely, for example, there are mentioned the compounds described in JP-A-2001-330725, [0028] to [0056].

The polymer that may be used along with the discotic liquid-crystalline molecules is preferably one capable of changing the tilt angle of the discotic liquid-crystalline molecules.

Examples of the polymer are cellulose esters. Preferred examples of cellulose esters are described in JP-A-2000-155216, [0178]. So as not to interfere with the alignment of the liquid-crystalline molecules in the layer, the amount of the polymer to be added to the layer is preferably from 0.1 to 10% by mass of the liquid-crystalline molecules, more preferably from 0.1 to 8% by mass.

Preferably, the discotic nematic liquid-crystal phase/solid phase transition temperature of the discotic liquid-crystalline molecules falls between 70 and 300° C., more preferably between 70 and 170° C.

### Formation of Optically-Anisotropic Layer:

The optically-anisotropic layer may be formed by applying a coating solution that contains liquid-crystalline molecules and optionally a polymerization initiator and other optional components mentioned below, on the alignment film.

The solvent to be used in preparing the coating solution is preferably an organic solvent. Examples of the organic solvent are amides (e.g., N,N-dimethylformamide), sulfoxides (e.g., dimethylsulfoxide), heterocyclic compounds (e.g., pyridine), hydrocarbons (e.g., benzene, hexane), alkyl halides (e.g., chloroform, dichloromethane, tetrachloroethane), esters (e.g., methyl acetate, butyl acetate), ketones (e.g., acetone, methyl ethyl ketone), ethers (e.g., tetrahydrofuran, 1,2-dimethoxyethane). Of those, preferred are alkyl halides and ketones. Two or more such organic solvents may be used as combined.

The coating solution may be applied onto the alignment film in any known method (e.g., wire bar coating, extrusion coating, direct gravure coating, reverse gravure coating, die coating).

The thickness of the optically-anisotropic layer is preferably from 0.1 to 20 μm, more preferably from 0.5 to 15 μm, most preferably from 1 to 10 μm.

### Fixation of Alignment State of Liquid-Crystalline Molecules:

The aligned liquid-crystalline molecules may be fixed as they are in an alignment state. Preferably, the fixation is effected through polymerization. The polymerization includes thermal polymerization with a thermal polymerization initiator and optical polymerization with an optical polymerization initiator. Preferred is optical polymerization.

The optical polymerization initiator includes, for example, α-carbonyl compounds (as in U.S. Pat. Nos. 2,367,661, 2,367,670), acyloin ethers (as in U.S. Pat. No. 2,448,828), α-hydrocarbon-substituted aromatic acyloin compounds (as in U.S. Pat. No. 2,722,512), polynuclear quinone compounds (as in U.S. Pat. Nos. 3,046,127, 2,951,758), combination of triarylimidazole dimer and p-aminophenylketone (as in U.S. Pat. No. 3,549,367), acridine compounds and phenazine compounds (as in JP-A-60-105667, U.S. Pat. No. 4,239,850), and oxadiazole compounds (as in U.S. Pat. No. 4,212,970).

The amount of the optical polymerization initiator to be added is preferably from 0.01 to 20% by mass of the solid content of the coating solution, more preferably from 0.5 to 5% by mass.

Preferably, UV rays are used for light irradiation for polymerization of liquid-crystalline molecules. Preferably, the irradiation energy falls within a range of from 20 mJ/cm² to 50 J/cm², more preferably from 20 to 5000 mJ/cm², even more preferably from 100 to 800 mJ/cm². For promoting the optical polymerization, the light irradiation may be effected under heat.

A protective layer may be provided on the optically-anisotropic layer.

Preferably, the optical compensatory film may be combined with a polarizing film. Concretely, the above-mentioned optically-anisotropic layer-coating solution is applied onto the surface of a polarizing film to from an optically-anisotropic layer thereon. As a result, no polymer film exists between the polarizing film and the optically-anisotropic layer, and a thin polarizer is thus constructed of which the stress (strain×cross section×elasticity) to be caused by the dimensional change of the polarizing film is reduced. When the polarizer of the invention is fitted to large-size liquid-crystal display devices, then it does not produce a problem of light leakage and the devices can display high-quality images.

Preferably, the polarizing film and the optically-compensatory layer are so stretched that the tilt angle between the two may correspond to the angle formed by the transmission axis of the two polarizers to be stuck to both sides of the liquid crystal cell to constitute LCD, and the machine direction or the transverse direction of the liquid crystal cells. In general, the tilt angle is 45°. Recently, however, some devices in which the tile angle is not always 45° have been developed for transmission-type, reflection-type or semi-transmission-type LCDs, and it is desirable that the stretching direction is varied in any desired manner depending on the plan of LCDs.

### (3) Formation of Antireflection Layer (for Antireflection Film):

In general, an antireflection film is constructed by forming a low-refractivity layer that functions as a stain-preventing layer, and at least one layer having a higher refractivity than that of the low-refractivity layer (high-refractivity layer or middle-refractivity layer) on a transparent substrate.

A multi-layer film is formed by laminating transparent thin films of inorganic compounds (e.g., metal oxides) having a different refractivity, for example, in a mode of chemical vapor deposition (CVD) or physical vapor deposition (PVD); or a film of colloidal metal oxide particles is formed according to a sol-gel process with a metal compound such as a metal oxide, and then this is post-treated (e.g., UV irradiation as in JP-A-9-157855, or plasma treatment as in JP-A-2002-327310) to give a thin film.

On the other hand, various types of antireflection films of high producibility are proposed, which are formed by laminating thin films of inorganic particles dispersed in a matrix.

The antireflection films produced according to the above-mentioned coating methods may be further processed so that the surface of the outermost layer thereof is roughened to have an antiglare property.

The cellulose acylate film of the invention may be applied to any type as above. Especially preferably, the film is applied to film construction in a layers-coating system (layers-coated films).

### Layer Constitution of Layers-Coated Antireflection Film:

The antireflection film having a layer constitution of at least a middle-refractivity layer, a high-refractivity layer and a low-refractivity layer (outermost layer) formed in that order on a substrate is so planned that it satisfies the refractivity profile mentioned below.

Refractivity of high-refractivity layer>refractivity of middle-refractivity layer>refractivity of transparent support>refractivity of low-refractivity layer.

A hard coat layer may be disposed between the transparent support and the middle-refractivity layer. Further, the film may comprise a middle-refractivity hard coat layer, a high-refractivity layer and a low-refractivity layer.

For example, JP-A-8-122504, JP-A-8-110401, JP-A-10-300902, JP-A-2002-243906, JP-A-2000-111706 are referred to.

The constitutive layers may have other functions. For example, there are mentioned a stain-resistant low-refractivity layer and an antistatic high-refractivity layer (for example, as in JP-A-10-206603, JP-A-2002-243906).

Preferably, the haze of the antireflection film is at most 5%, more preferably at most 3%. Also preferably, the strength of the film is at least 1H measured in the pencil hardness test according to JIS K5400, more preferably at least 2H, most preferably at least 3H.

### High-Refractivity Layer and Middle-Refractivity Layer:

The high-refractivity layer of the antireflection film is formed of a cured film that contains at least ultrafine particles of an inorganic compound of high refractivity having a mean particle size of at most 100 nm and a matrix binder.

The high-refractivity inorganic compound particles are those of an inorganic compound having a refractivity of at least 1.65, preferably at least 1.9. The inorganic compound particles are, for example, those of a metal oxide with any of Ti, Zn, Sb, Sn, Zr, Ce, Ta, La and In, and those of a composite oxide with such metal atoms.

For example, the ultrafine particles may be processed with a surface-treating agent (e.g., silane coupling agent as in JP-A-11-295503, JP-A-11-153703, JP-A-2000-9908; anionic compound or organic metal coupling agent as in JP-A-2001-310432); or they may have a core/shell structure in which the core is a high-refractivity particle (e.g., as in JP-A-2001-166104); or they may be combined with a specific dispersant (e.g., as in JP-A-11-153703, U.S. Pat. No. 6,210,858 B1, JP-A-2002-2776069).

The material to from the matrix may be any known thermoplastic resin or curable resin film.

For the material, also preferred is at least one composition selected from a polyfunctional compound-containing composition in which the compound has at least two radical-polymerizing and/or cationic-polymerizing groups, and a composition of a hydrolyzing group-containing organic metal compound or its partial condensate. For it, for example, referred to are the compounds described in JP-A-2000-47004, JP-A-2001-315242, JP-A-2001-31871 and JP-A-2001-296401.

Also preferred is a curable film formed of a colloidal metal oxide obtained from a hydrolyzed condensate of a metal alkoxide, and a metal alkoxide composition. For example, it is described in JP-A-2001-293818.

The refractivity of the high-refractivity layer is generally from 1.70 to 2.20. Preferably, the thickness of the high-refractivity layer is from 5 nm to 10 μm, more preferably from 10 nm to 1 μm.

The refractivity of the middle-refractivity layer is so controlled that it may be between the refractivity of the low-refractivity layer and that of the high-refractivity layer. Preferably, the refractivity of the middle-refractivity layer is from 1.50 to 1.70.

### Low-Refractivity Layer:

The low-refractivity layer is laminated on the high-refractivity layer in order. The refractivity of the low-refractivity layer may be, for example, from 1.20 to 1.55, but preferably from 1.30 to 1.50.

Preferably, the low-refractivity layer is constructed as the outermost layer having good scratch resistance and good stain resistance. For significantly increasing the scratch resistance of the layer, it is effective to lubricate the surface of the layer. For it, for example, employable is a method of forming a thin layer that contains a conventional silicone compound or fluorine-containing compound introduced thereinto.

Preferably, the refractivity of the fluorine-containing compound is from 1.35 to 1.50, more preferably from 1.36 to 1.47. Also preferably, the fluorine-containing compound has a crosslinking or polymerizing functional group that contains a fluorine atom in an amount of from 35 to 80% by mass.

For example, herein usable are the compounds described in JP-A-9-222503, [0018] to [0026]; JP-A-11-38202, [0019] to [0030]; JP-A-2001-40284, [0027] to [0028]; JP-A-2000-284102.

Preferably, the silicone compound has a polysiloxane structure in which the polymer chain contains a curable functional group or a polymerizing functional group, and it forms a film having a crosslinked structure therein. For example, it includes reactive silicones (e.g., Silaplane by Chisso), and polysiloxanes double-terminated with a silanol group (as in JP-A-11-258403).

Preferably, the crosslinking or polymerizing group-having, fluorine-containing and/or siloxane polymer is crosslinked or polymerized simultaneously with or after the coating operation with the coating composition to form the outermost layer that contains a polymerization initiator and a sensitizer, by exposing the coating layer to light or heat.

Also preferred is a sol-gel curable film which comprises an organic metal compound such as a silane coupling agent and a specific fluorine-containing hydrocarbon group-having silane coupling agent and in which they are condensed in the presence of a catalyst to cure the film.

For example, there are mentioned a polyfluoroalkyl group-containing silane compound or its partial hydrolyzed condensate (as in JP-A-58-142958, JP-A-58-147483, JP-A-58-147484, JP-A-9-157582, JP-A-11-106704), and a silyl compound having a fluorine-containing long-chain group, poly(perfluoroalkylether) group (as in JP-A-2000-117902, JP-A-2001-48590, JP-A-2002-53804).

As other additives than the above, the low-refractivity layer may contain a filler (e.g., low-refractivity inorganic compound of which the primary particles have a mean particle size of from 1 to 150 nm, such as silicon dioxide (silica), fluorine-containing particles (magnesium fluoride, calcium fluoride, barium fluoride); organic fine particles described in JP-A-11-3820, [0020] to [0038]), a silane coupling agent, a lubricant, a surfactant, etc.

When the low-refractivity layer is positioned below an outermost layer, then it may be formed according to a vapor-phase process (e.g., vacuum evaporation, sputtering, ion plating, plasma CVD). However, a coating method is preferred as it produces the layer at low costs.

Preferably, the thickness of the low-refractivity layer is from 30 to 200 nm, more preferably from 50 to 150 nm, most preferably from 60 to 120 nm.

### Hard Coat Layer:

A hard coat layer may be disposed on the surface of a transparent support for increasing the physical strength of the antireflection film to be thereon. In particular, the layer is preferably disposed between a transparent support and the above-mentioned high-refractivity layer.

Also preferably, the hard coat layer is formed through crosslinking or polymerization of an optical and/or thermal curable compound. The curable functional group is preferably a photopolymerizing functional group, and the hydrolyzing functional group-containing organic metal compound is preferably an organic alkoxysilyl compound.

Specific examples of the compounds may be the same as those mentioned hereinabove for the high-refractivity layer.

Specific examples of the constitutive composition for the hard coat layer are described in, for example, JP-A-2002-144913, JP-A-2000-9908, and WO00/46617.

The high-refractivity layer may serve also as a hard coat layer. In such a case, it is desirable that fine particles are added to and finely dispersed in the hard coat layer in the same manner as that mentioned hereinabove for the formation of the high-refractivity layer.

Containing particles having a mean particle size of from 0.2 to 10 μm, the hard coat layer may serve also as an antiglare layer (this will be mentioned hereinunder) having an antiglare function.

The thickness of the hard coat layer may be suitably determined in accordance with the use thereof. Preferably, for example, the thickness of the hard coat layer is from 0.2 to 10 μm, more preferably from 0.5 to 7 μm.

Preferably, the strength of the hard coat layer is at least 1H as measured in the pencil hardness test according to JIS K5400, more preferably at least 2H, most preferably at least 3H. Also preferably, the abrasion of the test piece of the layer before and after the taper test according to JIS K5400 is as small as possible.

### Front-Scattering Layer:

A front-scattering layer may be provided for improving the viewing angle on the upper and lower sides and on the right and left sides of liquid-crystal display devices to which the film is applied. Fine particles having a different refractivity may be dispersed in the hard coat layer, and the resulting hard coat layer may serve also as a front-scattering layer.

For it, for example, referred to are JP-A-11-38208 in which the front-scattering coefficient is specifically defined; JP-A-2000-199809 in which the relative refractivity of transparent resin and fine particles is defined to fall within a specific range; and JP-A-2002-107512 in which the haze value is defined to be at least 40%.

### Other Layers:

In addition to the above-mentioned layers, the film may further has a primer layer, an antistatic layer, an undercoat layer, a protective layer, etc.

### Coating Method:

The constitutive layers of the antireflection film may be formed in various coating methods of, for example, dip coating, air knife coating, curtain coating, roller coating, wire bar coating, gravure coating, microgravure coating or extrusion coating (as in U.S. Pat. No. 2,681,294).

### Antiglare Function:

The antireflection film may have an antiglare function of scattering external light. The film may have the antiglare function by roughening its surface. When the antireflection film has the antiglare function, then its haze is preferably from 3 to 30%, more preferably from 5 to 20%, most preferably from 7 to 20%.

For roughening the surface of the antireflection film, employable is any method in which the roughened surface profile may be kept well. For example, there are mentioned a method of adding fine particles to a low-refractivity layer so as to roughen the surface of the layer (e.g., as in JP-A-2000-271878); a method of adding a small amount (from 0.1 to 50% by mass) of relatively large particles (having a particle size of from 0.05 to 2 μm) to the lower layer (high-refractivity layer, middle-refractivity layer or hard coat layer) below a low-refractivity layer to thereby roughen the surface of the lower layer, and forming a low-refractivity layer on it while keeping the surface profile of the lower layer (e.g., as in JP-A-2000-281410, JP-A-2000-95893, JP-A-2001-100004, JP-A-2001-281407); and a method of physically transferring a roughened profile onto the surface of the outermost layer (stain-resistant layer) (for example, according to embossing treatment as in JP-A-63-278839, JP-A-11-183710, JP-A-2000-275401).

### Liquid-Crystal Display Device:

The cellulose acylate film of the invention may be favorably used in liquid-crystal display devices. In particular, the cellulose acylate film of the invention is effective when used as an optical compensatory sheet in liquid-crystal display devices. In case where the film itself is used as an optical compensatory sheet, then a polarizing element (mentioned below) and the optical compensatory sheet formed of the cellulose acylate film are preferably so disposed that the transmission axis of the former could be substantially in parallel or vertical to the slow axis of the latter. The configuration of the polarizing element and the optical compensatory sheet of the type is described in JP-A-10-48420. A liquid-crystal display device comprises a liquid-crystal cell that carries liquid crystal between two electrode substrates, two polarizing elements each disposed on both sides of the liquid-crystal cell, and at least one optical compensatory sheet disposed between the liquid-crystal cell and the polarizing element.

Various types of liquid-crystal display devices to which the cellulose acylate film of the invention is applicable are described below.

### TN-Mode Liquid-Crystal Display Device:

A TN-mode is most popularly utilized in color TFT liquid-crystal display devices, and this is described in a large number of references. The alignment state in the liquid-crystal cell at the time of black level of TN-mode display is as follows: The rod-shaped liquid-crystalline molecules stand up in the center of the cell, and they lie down at around the substrate of the cell.

### OCB-Mode Liquid-Crystal Display Device:

This is a bent-alignment mode liquid-crystal cell in which the rod-shaped liquid-crystalline molecules are aligned substantially in the opposite directions (symmetrically) between the upper part and the lower part of the liquid-crystal cell. The liquid-crystal display device that comprises such a bent-alignment mode liquid-crystal cell is disclosed in U.S. Pat. Nos. 4,583,825 and 5,410,422. In this, since the rod-shaped liquid-crystalline molecules are symmetrically aligned in the upper part and the lower part of the liquid-crystal cell, the bent-alignment mode liquid-crystal cell has a self-optically-compensatory function. Accordingly, the liquid-crystal mode of the type is referred to as an OCB (optically-compensatory bent) liquid-crystal mode.

Regarding the alignment state at the time of black level of display in the OCB-mode liquid-crystal cell, the rod-shaped liquid-crystalline molecules stand up in the center of the cell, and they lie down at around the substrate of the cell, like in the TN-mode liquid-crystal cell.

### VA-Mode Liquid-Crystal Display Device:

This is characterized in that the rod-shaped liquid-crystalline molecules therein are substantially vertically aligned in the absence of voltage application thereto. The VA-mode liquid-crystal cell includes (1) a VA-mode liquid-crystal cell in the narrow sense of the word, in which the rod-shaped liquid-crystalline molecules are substantially vertically aligned in the absence of voltage application thereto but are substantially horizontally aligned in the presence of voltage application thereto (as in JP-A-2-176625), further including in addition to it, (2) a multi-domain VA-mode (MVA-mode) liquid crystal cell for viewing angle expansion (as in SID97, Digest of Tech. Papers (preprint), 28 (1997) 845), (3) an n-ASM-mode liquid-crystal cell in which the rod-shaped liquid-crystalline molecules are substantially vertically aligned in the absence of voltage application thereto but are subjected to twisted multi-domain alignment in the presence of voltage application thereto (as in the preprint in the Nippon Liquid Crystal Discussion Meeting, 58-59 (1998)), and (4) a SURVAIVAL-mode liquid-crystal cell (as announced in LCD International 98).

### Other Liquid-Crystal Display Devices:

ECB-mode and STN-mode liquid-crystal display devices may be optically compensated in the same consideration as above.

### Analytic Method and Evaluation Method:

Methods for analyzing and evaluating cellulose acylate grains and cellulose acylate film are described below. The data found herein are determined according to the methods mentioned below.

### (1) Heat of Crystalline Fusion:

From 10 to 20 mg of cellulose acylate grains are weighed and put into a sample pan. Using DSC (differential scanning calorimeter), the sample is heated from room temperature up to 250° C. at a rate of 10° C./min, and the heat of crystalline fusion of the sample (J/g) is obtained from the sum total of the areas of the heat absorption peaks appearing between 170° C. and 250° C. In the invention, when the absorption peak is not detected, the heat of crystalline fusion is expressed as 0 (J/g).

### (2) Acicular Impurities:

Cellulose acylate grains are dissolved in dichloromethane to have a concentration of 20% by mass, and the solution is cast to form a cellulose acylate film having a thickness of 100 μm. This is set on a polarization microscope and observed at 50-power. The acicular impurities are seen as brightening spots under a cross-Nicol condition, and the number of the brightening spots is counted per the unit weight of the sample.

### (3) Sulfate Group Content:

The sulfate group content of cellulose acylate grains is determined according to ASTM D-817-96.

### (4) Alkali Metal Amount and Group-2 Metal Amount:

Nitric acid is added to cellulose acylate grains and a shed with multiwaves, and then dissolved in water. According to an ICP-OES method, the alkali metal amount and the Group-2 metal amount in the sample are determined.

### (5) Weight-Average Degree of Polymerization (DPw):

Cellulose acylate is dissolved in THF to prepare a sample solution (0.5% by mass). Under the condition mentioned below, the weight-average molecular weight (Mw) of the sample is determined through GPC. A calibration curve is drawn, using polystyrene (TSK-standard polystyrene, having a molecular weight of 1050, 5970, 18100, 37900, 190000, 706000). The thus-found value Mw is divided by the molecular weight per one segment obtained from the degree of substitution determined according to the method mentioned below, and this is DPw.

### Column: TSK GEL Super HZ4000, TSK GEL Super HZ2000

TSK GEL Super HZM-M, TSK Guard Column Super HZ-L.

### Column Temperature: 40° C.

### Eluent: THF.

Flow Rate: 1 ml/min.

### Detector: R1.

### (6) Degree of Substitution for Acyl Group in Cellulose Acylate:

According to ASTM D-817-91, cellulose acylate is completely hydrolyzed, and the resulting free carboxylic acid or its salt is quantitatively determined through gas chromatography or liquid chromatography to obtain the degree of substitution for an acyl group in the sample.

### (7) SP Value:

The data described in J. Brandrup, E. H. Immergut and E. A. Grulke, “Polymer Handbook Fourth Edition”, VII/688-694 (1998), John Wiley & Sons, Inc. are referred to herein. The others not described in this may be obtained in the manner mentioned below (value at 298° K).

According to the method described in J. H. Hildebrand, “Solubility of Nonelectrolytes”, 424-427 (1950), Reinhold Publishing Co., the data are obtained according to the following formula (1):

SP Value (σ)=[(ΔH−RT)/VL]^{1/2} (1)

wherein σ represents a solubility parameter; ΔH represents a heat of evaporation; VL represents a molar volume; R represents a vapor constant (1.986 cal/mol).

ΔH is a value at 298° K computed as in the following formula (2) based on the boiling point of the sample, according to the Hildebrand rule. Regarding the method of computation of the solubility parameter according to the Hildebrand rule, referred to is the description in J. H. Hildebrand, “Solubility of Nonelectrolytes”, 424-427 (1950), Reinhold Publishing Co.

ΔH298=23.7Tb+0.020Tb²−2950 (2)

wherein Tb represents the boiling point of the sample.

### EXAMPLES

The invention is described more concretely with reference to the following Examples and Comparative Examples. In the following Examples, the material used, its amount and ratio, the details of the treatment and the treatment process may be suitably modified or changed not overstepping the sprit and the scope of the invention. Accordingly, the invention should not be limited to the Examples mentioned below.

### Example A

### 1. Production of Cellulose Acylate

(1) Production of Cellulose Acetate Propionate (Examples 1 to 44, 49):

### A) Activation:

80 parts by mass of cellulose (pulp) and 33 parts by mass of acetic acid were put into a reactor equipped with a stirring device and a cooling device, and heated at 60° C. for 4 hours to activate the cellulose. The activation time was changed to produce cellulose acylates of Examples 17 to 19 having a different acicular impurity content.

### B) Acylation:

32 parts by mass of acetic anhydride, 540 parts by mass or propionic acid, 558 parts by mass of propionic anhydride and 4 parts by mass of sulfuric acid were mixed and cooled to −20° C., and added to the reactor. The cellulose was esterified under such control that the highest reaction temperature could be 35° C., and the time when the viscosity of the reaction liquid reached 910 cP is the end point of the reaction. The reaction mixture was so controlled that its temperature at the end point could be 15° C. A reaction stopper prepared by mixing 133 parts by mass of water and 133 parts by mass of acetic acid and cooled to −5° C. was added to the reaction mixture so that the temperature of the reaction mixture could not be higher than 23° C. The blend ratio of the reactants was varied to obtain cellulose acylates of Examples 39 to 44.

### C) Partial Hydrolysis:

The reaction mixture was stirred at 60° C. for 2 hours for partial hydrolysis, and then filtered through filter paper. The stirring time was changed to obtain cellulose acylates of Examples 35 to 38 having a different degree of polymerization.

### D) Control of Sulfuric Acid Amount:

Next, a mixed solution of 77 g (2 equivalents to sulfuric acid) of magnesium acetate 4-hydrate, 77 g of acetic acid and 77 g of water was added to the reactor (neutralization), and stirred at 60° C. for 2 hours (post-heating). This was filtered through filter paper, and then mixed with an aqueous acetic acid solution for reprecipitation of the resulting polymer compound, and then repeatedly washed with hot water at 70 to 80° C. The washing time was changed to obtain cellulose acylates of Examples 20 to 29 in which the residual sulfuric acid amount differs.

### E) Addition of Alkali Metal, Group-2 Metal:

After dewatered, the cellulose acylate was dipped in an aqueous solution of an alkali metal or Group-2 metal compound described in Table 1, and stirred for 30 minutes. Next, this was again dewatered. This was dried in vacuum at 60° C. for 12 hours. The type of the alkali metal or Group-2 metal compound and the concentration of the aqueous solution thereof were varied to give M/S, or that is, (sum of the molar amount of alkali metal and the molar amount of Group-2 metal)/(the molar amount of sulfate group) as in Table 1.

(2) Production of Cellulose Acetate Butyrate, Etc. (Examples 45 to 48):

### A) Activation:

200 parts by mass of cellulose (linter) and 100 parts by mass of acetic acid were put into a reactor equipped with a stirring device and a cooling device, and heated at 60° C. for 4 hours to activate the cellulose.

### B) Acylation:

161 parts by mass of acetic acid, 449 parts by mass of acetic anhydride, 742 parts by mass of butyric acid, 1349 parts by mass of butyric anhydride and 14 parts by mass of sulfuric acid were mixed and cooled to −20° C., and added to the reactor.

### C) Partial Hydrolysis:

The cellulose was esterified under such control that the highest reaction temperature could be 30° C. and the time when the viscosity of the reaction liquid reached 1050 cP is the end point of the reaction. The reaction mixture was so controlled that its temperature at the end point could be 10° C. A reaction stopper prepared by mixing 297 parts by mass of water and 558 parts by mass of acetic acid and cooled to −5° C. was added to the reaction mixture so that the temperature of the reaction mixture could not be higher than 23° C.

### D) Control of Sulfuric Acid Amount:

The reaction mixture was stirred at 60° C. for 2 hours and 30 minutes for partial hydrolysis, and then filtered through filter paper. This was mixed with aqueous acetic acid solution for reprecipitation of the resulting polymer compound, and then this was repeatedly washed with hot water at 70 to 80° C.

### E) Addition of Alkali Metal, Group-2 Metal:

After dewatered, the cellulose acylate was dipped in an aqueous solution of an alkali metal or Group-2 metal compound described in Table 1, and stirred for 30 minutes. In this state, the concentration of the aqueous solution was controlled to give the M/S ratio as in Table 1. Then, this was again dewatered. This was dried at 70° C. to obtain cellulose acylate butyrate (Example 45).

In the above-mentioned method of producing cellulose acylate, the time in A) activation was changed to control the amount of acicular impurities; the condition in B) acylation and that in C) partial hydrolysis were changed to control the degree of substitution and the molecular weight of the polymer; the post-heating time in D) sulfuric acid amount control was changed to control the sulfuric acid amount in the reaction system; and the condition (type, concentration) of the aqueous calcium hydroxide solution in E) addition of alkali metal, Group-2 metal was changed to control the alkali metal amount and the Group-2 metal amount in the polymer. Accordingly, different cellulose acylates (Examples 46 to 48) were produced.

### 2. Formation of Cellulose Acylate Pellets

The above cellulose acylate was dried at 120° C. for 3 hours to have a water content of 0.1% by mass, to which was added silicon dioxide particles (Aerosil R972V) in an amount of 0.05% by mass. Further, a stabilizer (Sumilizer GP by Sumitomo Chemical, 0.3% by mass), and a UV absorbent (Adekastab LA-31 by Asahi Denka Kogyo, 1% by mass) were added thereto. The resulting mixture was put into the hopper of a double-screw kneading extruder, and kneaded therein under the pelletization condition as in Table 1. The screw of the double-screw kneading extruder had a compression ratio of 3; the barrel diameter thereof was 40 mm; L/D=40; and the extrusion rate from the extruder was 150 kg/hr. After thus melted, the cellulose acylate was extruded out as strands having a diameter of 3 mm and solidified in water at 10° C., and then cut into pellets having a length of 5 mm. Thus produced, the pellets were dried at 100° C. for 10 minutes.

The pellets were analyzed for the heat quantity of crystalline fusion, the acicular impurities, the sulfate group content, the ratio of (sum of the molar amount of alkali metal and the molar amount of Group-2 metal)/(the molar amount of sulfate group) (this is M/S ratio in Table 1), the weight-average degree of polymerization (DPw) and the degree of substitution according to the methods mentioned above. The found data are given in Table 1.

### 3. Melt-Casting Film Formation

The above cellulose acylate pellets were dried in a vacuum drier at 110° C. for 2 hours so that they could have a residual water content of at most 0.01% by mass. These were put into the hopper controlled at (Tg−10)° C. of a double-screw extruder, and kneaded and melted therein in a nitrogen atmosphere. The first feed port temperature was 180° C.; the compression zone temperature was 210° C.; and the second feed port temperature was 220° C. The compression ratio of the fullflight screw was 4; and L (screw length)/D (screw diameter) was 30. At the outlet port of the extruder, the resin melt was filtered through a breaker plate-type filter, then led to pass through a gear pump, and again filtered through a 4-μm stainless leaf-type disc filter device.

The resulting resin melt was extruded out through the T-die of the extruder, and then formed into a film using the touch roll described in Example 1 in JP-A-11-235747 under a linear pressure shown in Table 1. The casting roll and the touch roll had a diameter of 400 mm, and set at 120° C. After the film was peeled off from the casting roll, and trimmed at both edges (3% of the overall width at each edge). This was knurled to a width of 10 mm and a height of 50 μm at both edges, and then wound up at a speed of 30 m/min. Thus wound up, the film had a width of 1.5 m and a length of 3000 m.

TABLE 1

| | Cellulose Acytate | | | | | | | |
|---|---|---|---|---|---|---|---|---|
| | Degree of Substitution | | | | | | | |
| | acetyl | propionyl | butyryl | pentanoyl | hexanoyl | Y (total | | Weight Average |
| | group | group | group | group | group | of Y1 to | | Degree of |
| | (X) | (Y1) | (Y2) | (Y3) | (Y4) | Y4) | X + Y | Polymerization |
| Example 1 | 0.4 | 2.5 | | | | 2.5 | 2.9 | 400 |
| Comparative Example 1 | 0.4 | 2.5 | | | | 2.5 | 2.9 | 400 |
| Example 2 | 0.4 | 2.5 | | | | 2.5 | 2.9 | 180 |
| Example 3 | 0.4 | 2.5 | | | | 2.5 | 2.9 | 480 |
| Comparative Example 2 | 0.4 | 2.5 | | | | 2.5 | 2.9 | 530 |
| Comparative Example 3 | 0.4 | 2.5 | | | | 2.5 | 2.9 | 400 |
| Example 4 | 0.4 | 2.5 | | | | 2.5 | 2.9 | 400 |
| Example 5 | 0.4 | 2.5 | | | | 2.5 | 2.9 | 400 |
| Comparative Example 4 | 0.4 | 2.5 | | | | 2.5 | 2.9 | 400 |
| Example 6 | 0.4 | 2.5 | | | | 2.5 | 2.9 | 400 |
| Example 7 | 0.4 | 2.5 | | | | 2.5 | 2.9 | 400 |
| Example 8 | 0.4 | 2.5 | | | | 2.5 | 2.9 | 400 |
| Example 9 | 0.4 | 2.5 | | | | 2.5 | 2.9 | 400 |
| Example 10 | 0.4 | 2.5 | | | | 2.5 | 2.9 | 400 |
| Example 11 | 0.4 | 2.5 | | | | 2.5 | 2.9 | 400 |
| Example 12 | 0.4 | 2.5 | | | | 2.5 | 2.9 | 400 |
| Example 13 | 0.4 | 2.5 | | | | 2.5 | 2.9 | 400 |
| Example 14 | 0.4 | 2.5 | | | | 2.5 | 2.9 | 400 |
| Example 15 | 0.4 | 2.5 | | | | 2.5 | 2.9 | 400 |
| Example 16 | 0.4 | 2.5 | | | | 2.5 | 2.9 | 400 |
| Example 17 | 0.4 | 2.5 | | | | 2.5 | 2.9 | 400 |
| Example 18 | 0.4 | 2.5 | | | | 2.5 | 2.9 | 400 |
| Example 19 | 0.4 | 2.5 | | | | 2.5 | 2.9 | 400 |
| Example 20 | 0.4 | 2.5 | | | | 2.5 | 2.9 | 400 |
| Example 21 | 0.4 | 2.5 | | | | 2.5 | 2.9 | 400 |
| Example 22 | 0.4 | 2.5 | | | | 2.5 | 2.9 | 400 |
| Example 23 | 0.4 | 2.5 | | | | 2.5 | 2.9 | 400 |
| Example 24 | 0.4 | 2.5 | | | | 2.5 | 2.9 | 400 |
| Example 25 | 0.4 | 2.5 | | | | 2.5 | 2.9 | 400 |
| Example 26 | 0.4 | 2.5 | | | | 2.5 | 2.9 | 400 |
| Example 27 | 0.4 | 2.5 | | | | 2.5 | 2.9 | 400 |
| Example 28 | 0.4 | 2.5 | | | | 2.5 | 2.9 | 400 |
| Example 29 | 0.4 | 2.5 | | | | 2.5 | 2.9 | 400 |
| Example 30 | 0.4 | 2.5 | | | | 2.5 | 2.9 | 400 |
| Example 31 | 0.4 | 2.5 | | | | 2.5 | 2.9 | 400 |
| Example 32 | 0.4 | 2.5 | | | | 2.5 | 2.9 | 400 |
| Example 33 | 0.4 | 2.5 | | | | 2.5 | 2.9 | 400 |
| Example 34 | 0.4 | 2.5 | | | | 2.5 | 2.9 | 400 |
| Example 35 | 0.4 | 2.5 | | | | 2.5 | 2.9 | 220 |
| Example 36 | 0.4 | 2.5 | | | | 2.5 | 2.9 | 260 |
| Example 37 | 0.4 | 2.5 | | | | 2.5 | 2.9 | 480 |
| Example 38 | 0.4 | 2.5 | | | | 2.5 | 2.9 | 530 |
| Example 39 | 0.05 | 2.95 | | | | 2.95 | 3.0 | 450 |
| Example 40 | 0.8 | 2.0 | | | | 2.0 | 2.8 | 450 |
| Example 41 | 1.6 | 1.2 | | | | 1.2 | 2.8 | 450 |
| Example 42 | 2.0 | 0.8 | | | | 0.8 | 2.8 | 450 |
| Example 43 | 0.6 | 2.0 | | | | 2.0 | 2.6 | 450 |
| Example 44 | 0.4 | 2.0 | | | | 2.0 | 2.4 | 450 |
| Example 45 | 1.5 | | 1.2 | | | 1.2 | 2.7 | 450 |
| Example 46 | 1.0 | | 1.7 | | | 1.7 | 2.7 | 450 |
| Example 47 | 0.4 | | 2.5 | | | 2.5 | 2.9 | 450 |
| Example 48 | 1.0 | 1.2 | 0.2 | 0.2 | 0.2 | 1.8 | 2.8 | 450 |
| Example 49 | 0.2 | 2.5 | | | | 2.5 | 2.7 | 270 |
| Comparative Example 5 | 0.2 | 2.5 | | | | 2.5 | 2.7 | 200 |

| | | Pelletization | | | | | | |
|---|---|---|---|---|---|---|---|---|
| | | | | | Vacuum | | | Properties |
| | | | | | Degassing | | | of Pellets |
| | Cellulose | | Resin | | (pressure in | | | Heat of |
| | Acytate | Screw | Kneading | Kneading | kneading) | | Residual | Crystalline |
| | Tg | Revolution | Pressure | Temperature | (atmospheric | Inert | Oxygen | Fusion |
| | (° C.) | (rpm) | (MPa) | (° C.) | pressure) | Gas | (%) | (J/g) |
| Example 1 | 135 | 150 | 5 | 200 | 0.5 | N₂ | 5 | 0.2 |
| Comparative Example 1 | 135 | 100 | 1 | 190 | 0.3 | He | 3 | 15 |
| Example 2 | 135 | 100 | 2 | 190 | 0.3 | He | 3 | 3 |
| Example 3 | 135 | 100 | 9 | 190 | 0.3 | He | 3 | 4 |
| Comparative Example 2 | 135 | 100 | 11 | 190 | 0.3 | He | 3 | 16 |
| Comparative Example 3 | 135 | 40 | 5 | 190 | 0.3 | He | 3 | 12 |
| Example 4 | 135 | 60 | 5 | 190 | 0.3 | He | 3 | 2 |
| Example 5 | 135 | 280 | 5 | 190 | 0.3 | He | 3 | 3 |
| Comparative Example 4 | 135 | 330 | 5 | 190 | 0.3 | He | 3 | 12 |
| Example 6 | 135 | 150 | 5 | 150 | 0.5 | Ar | 10 | 5 |
| Example 7 | 135 | 150 | 5 | 170 | 0.5 | Ar | 10 | 1 |
| Example 8 | 135 | 150 | 5 | 220 | 0.5 | Ar | 10 | 1 |
| Example 9 | 135 | 150 | 5 | 230 | 0.5 | Ar | 10 | 5 |
| Example 10 | 135 | 150 | 5 | 200 | 1 | — | 19 | 4 |
| Example 11 | 135 | 150 | 5 | 200 | 0.9 | — | 17 | 2.5 |
| Example 12 | 135 | 150 | 5 | 200 | 0 | — | 2 | 1.5 |
| Example 13 | 135 | 150 | 5 | 200 | 1 | N₂ | 19 | 5 |
| Example 14 | 135 | 150 | 5 | 200 | 1 | N₂ | 17 | 2.3 |
| Example 15 | 135 | 150 | 5 | 200 | 1 | N₂ | 2 | 1 |
| Example 16 | 135 | 150 | 5 | 200 | 0.9 | N₂ | 17 | 0.5 |
| Example 17 | 135 | 150 | 5 | 200 | 0.5 | N₂ | 5 | 6 |
| Example 18 | 135 | 150 | 5 | 200 | 0.5 | N₂ | 5 | 2 |
| Example 19 | 135 | 150 | 5 | 200 | 0.5 | N₂ | 5 | 0.8 |
| Example 20 | 135 | 150 | 5 | 200 | 0.5 | N₂ | 5 | 6 |
| Example 21 | 135 | 150 | 5 | 200 | 0.5 | N₂ | 5 | 2.2 |
| Example 22 | 135 | 150 | 5 | 200 | 0.5 | N₂ | 5 | 1.5 |
| Example 23 | 135 | 150 | 5 | 200 | 0.5 | N₂ | 5 | 1.5 |
| Example 24 | 135 | 150 | 5 | 200 | 0.5 | N₂ | 5 | 5 |
| Example 25 | 135 | 150 | 5 | 200 | 0.5 | N₂ | 5 | 2.3 |
| Example 26 | 135 | 150 | 5 | 200 | 0.5 | N₂ | 5 | 1.5 |
| Example 27 | 135 | 150 | 5 | 200 | 0.5 | N₂ | 5 | 4.5 |
| Example 28 | 135 | 150 | 5 | 200 | 0.5 | N₂ | 5 | 0.6 |
| Example 29 | 135 | 150 | 5 | 200 | 0.5 | N₂ | 5 | 2.8 |
| Example 30 | 135 | 150 | 5 | 200 | 0.5 | N₂ | 5 | 0.2 |
| Example 31 | 135 | 150 | 5 | 200 | 0.5 | N₂ | 5 | 0.2 |
| Example 32 | 135 | 150 | 5 | 200 | 0.5 | N₂ | 5 | 0.2 |
| Example 33 | 135 | 150 | 5 | 200 | 0.5 | N₂ | 5 | 0.2 |
| Example 34 | 135 | 150 | 5 | 200 | 0.5 | N₂ | 5 | 0.2 |
| Example 35 | 133 | 150 | 5 | 200 | 0.5 | N₂ | 5 | 0.2 |
| Example 36 | 134 | 150 | 5 | 200 | 0.5 | N₂ | 5 | 0.2 |
| Example 37 | 135 | 150 | 5 | 200 | 0.5 | N₂ | 5 | 0.2 |
| Example 38 | 136 | 150 | 5 | 200 | 0.5 | N₂ | 5 | 0.2 |
| Example 39 | 130 | 150 | 5 | 200 | 0.5 | N₂ | 5 | 0.2 |
| Example 40 | 130 | 150 | 5 | 200 | 0.5 | N₂ | 5 | 0.2 |
| Example 41 | 140 | 150 | 5 | 200 | 0.5 | N₂ | 5 | 0.2 |
| Example 42 | 145 | 150 | 5 | 200 | 0.5 | N₂ | 5 | 0.2 |
| Example 43 | 132 | 150 | 5 | 200 | 0.5 | N₂ | 5 | 0.2 |
| Example 44 | 135 | 150 | 5 | 200 | 0.5 | N₂ | 5 | 0.2 |
| Example 45 | 135 | 150 | 5 | 200 | 0.5 | N₂ | 5 | 0.2 |
| Example 46 | 130 | 150 | 5 | 200 | 0.5 | N₂ | 5 | 0.2 |
| Example 47 | 125 | 150 | 5 | 200 | 0.5 | N₂ | 5 | 0.2 |
| Example 48 | 95 | 150 | 5 | 200 | 0.5 | N₂ | 5 | 0.2 |
| Example 49 | 140 | 150 | 5 | 200 | 0.5 | N₂ | 5 | 0 |
| Comparative Example 5 | 140 | 30 | 0 | 240 | 1 | — | 21 | 20 |

| | | | | | Film Formation | | Evaluation of Film | |
|---|---|---|---|---|---|---|---|---|
| | Properties of Pellets | | | | Linear | | Aged | |
| | Acicular | Sulfate | | | Pressure of | | Discoloration | |
| | Impurities | group | | | Touch Roll | Thickness | (400 nm | Yellowing |
| | (/mg] | (ppm) | M/S* | Type of M* | (kg/cm) | (μm) | absorbance) | In LCD |
| Example 1 | 0 | 80 | 0.6 | Ca(OH)₂ | 10 | 80 | 0.02 | 0 |
| Comparative Example 1 | 0 | 80 | 0.6 | Ca(OH)₂ | 10 | 80 | 0.36 | 9 |
| Example 2 | 0 | 80 | 0.6 | Ca(OH)₂ | 10 | 80 | 0.09 | 3 |
| Example 3 | 0 | 80 | 0.6 | Ca(OH)₂ | 10 | 80 | 0.10 | 3 |
| Comparative Example 2 | 0 | 80 | 0.6 | Ca(OH)₂ | 10 | 80 | 0.38 | 9 |
| Comparative Example 3 | 0 | 80 | 0.6 | Ca(OH)₂ | 10 | 80 | 0.39 | 8 |
| Example 4 | 0 | 80 | 0.6 | Ca(OH)₂ | 10 | 80 | 0.11 | 3 |
| Example 5 | 0 | 80 | 0.6 | Ca(OH)₂ | 10 | 80 | 0.09 | 4 |
| Comparative Example 4 | 0 | 80 | 0.6 | Ca(OH)₂ | 10 | 80 | 0.33 | 8 |
| Example 6 | 0 | 80 | 0.6 | Ca(OH)₂ | 10 | 80 | 0.15 | 5 |
| Example 7 | 0 | 80 | 0.6 | Ca(OH)₂ | 10 | 80 | 0.06 | 2 |
| Example 8 | 0 | 80 | 0.6 | Ca(OH)₂ | 10 | 80 | 0.07 | 2 |
| Example 9 | 0 | 80 | 0.6 | Ca(OH)₂ | 10 | 80 | 0.16 | 5 |
| Example 10 | 0 | 80 | 0.6 | Ca(OH)₂ | 10 | 80 | 0.15 | 5 |
| Example 11 | 0 | 80 | 0.6 | Ca(OH)₂ | 10 | 80 | 0.08 | 2 |
| Example 12 | 0 | 80 | 0.6 | Ca(OH)₂ | 10 | 80 | 0.06 | 2 |
| Example 13 | 0 | 80 | 0.6 | Ca(OH)₂ | 10 | 80 | 0.16 | 5 |
| Example 14 | 0 | 80 | 0.6 | Ca(OH)₂ | 10 | 80 | 0.09 | 2 |
| Example 15 | 0 | 80 | 0.6 | Ca(OH)₂ | 10 | 80 | 0.07 | 2 |
| Example 16 | 0 | 80 | 0.6 | Ca(OH)₂ | 10 | 80 | 0.03 | 0 |
| Example 17 | 60 | 80 | 0.6 | Mg(OH)₂ | 10 | 100 | 0.18 | 4 |
| Example 18 | 45 | 80 | 0.6 | Mg(OH)₂ | 10 | 100 | 0.11 | 1 |
| Example 19 | 20 | 80 | 0.6 | Mg(OH)₂ | 10 | 100 | 0.06 | 1 |
| Example 20 | 0 | 220 | 1.2 | Ca(OH)₂ | 10 | 80 | 0.15 | 5 |
| Example 21 | 0 | 180 | 1.2 | Ca(OH)₂ | 10 | 80 | 0.11 | 2 |
| Example 22 | 0 | 30 | 1.2 | Ca(OH)₂ | 10 | 80 | 0.08 | 1 |
| Example 23 | 0 | 0 | 1.2 | Ca(OH)₂ | 10 | 80 | 0.03 | 0 |
| Example 24 | 0 | 100 | 3.2 | Ca(OH)₂ | 10 | 80 | 0.14 | 5 |
| Example 25 | 0 | 100 | 2.8 | Ca(OH)₂ | 10 | 80 | 0.09 | 2 |
| Example 26 | 0 | 100 | 0.4 | Ca(OH)₂ | 10 | 80 | 0.05 | 1 |
| Example 27 | 0 | 100 | 0.2 | Ca(OH)₂ | 10 | 80 | 0.15 | 4 |
| Example 28 | 0 | 180 | 2.8 | Ca(OH)₂ | 10 | 80 | 0.03 | 1 |
| Example 29 | 0 | 220 | 3.2 | Ca(OH)₂ | 10 | 80 | 0.23 | 6 |
| Example 30 | 0 | 60 | 1.5 | NaH(CO₃) | not used | 80 | 0.18 | 4 |
| Example 31 | 0 | 60 | 1.5 | NaH(CO₃) | 2 | 80 | 0.14 | 2 |
| Example 32 | 0 | 60 | 1.5 | NaH(CO₃) | 4 | 80 | 0.05 | 1 |
| Example 33 | 0 | 60 | 1.5 | NaH(CO₃) | 90 | 80 | 0.05 | 1 |
| Example 34 | 0 | 60 | 1.5 | NaH(CO₃) | 120 | 80 | 0.12 | 2 |
| Example 35 | 0 | 40 | 0.6 | KH(CO₃) | 10 | 120 | 0.14 | 4 |
| Example 36 | 0 | 40 | 0.6 | KH(CO₃) | 10 | 120 | 0.05 | 1 |
| Example 37 | 0 | 40 | 0.6 | KH(CO₃) | 10 | 120 | 0.04 | 2 |
| Example 38 | 0 | 40 | 0.6 | KH(CO₃) | 10 | 120 | 0.19 | 5 |
| Example 39 | 0 | 150 | 2.2 | Ca(OH)₂ | 50 | 280 | 0.04 | 0 |
| Example 40 | 0 | 150 | 2.2 | Ca(OH)₂ | 50 | 280 | 0.08 | 1 |
| Example 41 | 0 | 150 | 2.2 | Ca(OH)₂ | 50 | 280 | 0.10 | 2 |
| Example 42 | 0 | 150 | 2.2 | Ca(OH)₂ | 50 | 280 | 0.18 | 4 |
| Example 43 | 0 | 150 | 2.2 | Ca(OH)₂ | 50 | 280 | 0.06 | 2 |
| Example 44 | 0 | 150 | 2.2 | Ca(OH)₂ | 50 | 200 | 0.19 | 4 |
| Example 45 | 0 | 150 | 2.2 | Ca(OH)₂ | 50 | 30 | 0.10 | 1 |
| Example 46 | 0 | 150 | 2.2 | Ca(OH)₂ | 50 | 30 | 0.06 | 1 |
| Example 47 | 0 | 150 | 2.2 | Ca(OH)₂ | 50 | 30 | 0.05 | 1 |
| Example 48 | 0 | 150 | 2.2 | Ca(OH)₂ | 50 | 30 | 0.12 | 2 |
| Example 49 | 0 | 90 | 1 | Ca(OH)₂ | 30 | 80 | 0.03 | 0 |
| Comparative Example 5 | 350 | 350 | 0 | — | not used | 80 | 0.55 | 10 |

*M: sum of molar amount of alkali metal and molar amount of alkaline earth metal.
S: molar amount of sulfate group.

### 4. Formation of Stretched Cellulose Acylate Film

The unstretched sheet was stretched at Tg+10° C. at 300%/min to the draw ratio mentioned below. Tg means the glass transition temperature of each film, and this is determined through DSC at 10° C./min, at which the base line in DSC begins to shift from the low-temperature side.

The stretched film was analyzed with an automatic birefringence meter (KOBRA-21ADH, by Oji Scientific Instruments) at 25° C. and 60% RH. The results of Example 1 are shown below, and the other Examples gave the same results.

TABLE 2

| | Draw Ratio in | Draw Ratio in | | |
|---|---|---|---|---|
| | MD-Stretching | CD-Stretching | RE | Rth |
| | (%) | (%) | (nm) | (nm) |
| Unstretched | 0 | 0 | 0 | 0 |
| Film | | | | |
| Stretched | 300 | 0 | 200 | 100 |
| Film 1 | | | | |
| Stretched | 50 | 10 | 60 | 220 |
| Film 2 | | | | |
| Stretched | 50 | 50 | 0 | 450 |
| Film 3 | | | | |
| Stretched | 50 | 10 | 60 | 220 |
| Film 4 | | | | |
| Stretched | 0 | 150 | 150 | 150 |
| Film 5 | | | | |

### 5. High-Temperature Aging of Unstretched or Stretched Cellulose Acylate Film

The cellulose acylate film was cut into sheets, and aged at 80° C. and 10% RH for 1000 hours (long-term aging). Next, its absorbance at 400 nm was measured, and this was converted into a value of the film having a thickness of 100 μm (found absorbance value×(100/actual thickness (μm)). This is the degree of yellowing of the film after long-term aging, and shown in Table 1 (in which this is in the column of aged discoloration).

### 6. Construction of Polarizer

(1) Saponification of Cellulose Acylate Film:

The long-term-aged unstretched cellulose acylate film and stretched acylate film were saponified by dipping in a saponification solution according to the process mentioned below. The same results were obtained when the film was coated with the saponification solution.

(1-1) Dipping Saponification:

An aqueous NaOH (1.5 mol/L) solution was prepared as a saponification solution, and conditioned at 60° C. The cellulose acylate film was dipped in the solution for 2 minutes. Next, this was dipped in an aqueous sulfuric acid (0.05 mol/L) solution for 30 seconds, and then led to pass through a water-washing bath.

(1-2) Coating Saponification:

20 parts by mass of water was added to 80 parts by mass of isopropanol, and KOH was dissolved therein to have a concentration of 1.5 mol/L. This was conditioned at 60° C. and used as a saponification solution. The saponification solution was applied to the cellulose acylate film at 60° C. in a degree of 10 g/m², and the film was thus saponified for 1 minute. Next, this was washed by spraying thereon hot water at 50° C. thereon in a degree of 10 L/m² min for 1 minute.

(2) Preparation of Polarizing Film:

According to Example 1 in JP-A-2001-141926, a film was stretched in the machine direction, between two pairs of nip rolls having a different peripheral speed to prepare a polarizing film having a thickness of 20 μm.

(3) Lamination:

Thus obtained, the polarizing film was laminated with any of the saponified, unstretched or stretched cellulose acylate film, using an aqueous 3% PVA (Kuraray's PVA-117H) solution as an adhesive, in such a manner that the polarization axis could cross the machine direction of the cellulose acylate film at 45 degrees. Thus constructed, the polarizer was fitted to a 20-inch VA-mode liquid-crystal display device of FIGS. 2 to 9 in JP-A-2000-154261, and the device was driven for entire white display. In this stage, the device panel was visually checked for yellowing, and the result is shown in Table 1 (yellowing in LCD). Each sample was evaluated at 10 points of scores. A score of 0 point was given to samples with no yellowing; and a score of 10 points was given to samples with strong yellowing. The practicable level is at most 6, preferably at most 4, more preferably at most 2, even more preferably at most 1, and most preferably 0.

The films of the invention all had good results. On the other hand, the comparative films much yellowed. In particular, as compared with the film of Example 3-1 in JP-A-2000-352620 (Comparative Example 5), the film of the invention corresponding to it (Example 49) was significantly bettered.

### 7. Construction of Optical Compensatory Film

(1) Unstretched Film:

When the unstretched cellulose acylate film of the invention was used in the first transparent support in Example 1 in JP-A-11-316378, then good optical compensatory films were produced.

(2) Stretched Cellulose Acylate Film:

When the stretched cellulose acylate film of the invention was used in place of the liquid-crystal layer-coated cellulose acetate film in Example 1 in JP-A-11-316378, then good optical compensatory films were produced. Similarly, when the stretched cellulose acylate film of the invention was used in place of the liquid-crystal layer-coated cellulose acetate film in Example 1 in JP-A-7-333433, then good optical compensatory filter films were produced.

### 8. Construction of Low-Refractivity Film

According to Example 47 in Hatsumei Kyokai Disclosure Bulletin (No. 2001-1745, published by the Hatsumei Kyokai on Mar. 15, 2001), the stretched or unstretched cellulose acylate film of the invention was used in construction of low-refractivity films, and the films had good optical properties.

### 9. Construction of Liquid-Crystal Display Device

The above polarizer of the invention was used in the liquid-crystal display device described in Example 1 in JP-A-10-48420; the discotic liquid-crystalline molecules-containing optically-anisotropic layer and the polyvinyl alcohol-coated alignment film described in Example 1 in JP-A-9-26572; the 20-inch VA-mode liquid-crystal display device of FIGS. 2 to 9 in JP-A-2000-154261; and the 20-inch OCB-mode liquid-crystal display device of FIGS. 10 to 15 in JP-A-2000-154261. The low-refractivity film of the invention was stuck to the outermost surface layer of these liquid-crystal display devices, and evaluated through visual observation. All these exhibited good visibility.

In place of the pellets of Examples 1 to 49, used were cellulose acylate grains prepared by grinding these pellets to have a size of from 1 to 10 mm³, and cellulose acylate films were produced from then according to the methods of Examples 1 to 49. These cellulose acylate films also had good results, like the films formed from the cellulose acylate pellets.

In addition, other films were produced in the same manner as in Example 1, to which, however, a plasticizer, dioctyl adipate was added in an amount of 4% by mass (Example 50), or the plasticizer 2 of formula I in JP-A-2000-352620 was added in an amount of 6% by mass (Example 51). These films were compared with the film of Example 1. The films of Examples 50 and 51 were the same as the film of example 1 in point of the heat of crystalline fusion, the acicular impurities, the aged discoloration and the yellowing in LCD; but in Example 51, the plasticizer deposited out on the casting roll after the film was formed to a length of 100 m; and in Example 52, the plasticizer deposited out on the casting roll after the film was formed to a length of 1000 m. These deposits caused transfer stains on the films. In Example 1, no film stain was seen even after the film was formed to a length of 10000 m or more.

### Example B

0.1 parts by mass of acetic acid and 2.7 parts by mass of propionic acid were sprayed onto 10 parts by mass of cellulose (hardwood pulp), and then stored at room temperature for 1 hour. Apart from this, a mixture of 1.2 parts by mass of acetic anhydride, 61 parts by mass of propionic anhydride and 0.7 parts by mass of sulfuric acid was prepared, cooled to −10° C., and then mixed with the above pretreated cellulose in a reactor. After 30 minutes, the outer temperature was elevated up to 30° C., and the compounds were reacted for 4 hours. 46 parts by mass of aqueous 25% acetic acid solution was added to the reactor, and the inner temperature was elevated up to 60° C., and this was stirred for 2 hours. 6.2 parts by mass of a solution prepared by mixing magnesium acetate 4-hydrate, acetic acid and water (1/1/1 by weight) was added to it, and stirred for 30 minutes. The reaction liquid was filtered under pressure through metal sintered filters having a retention particle size of 40 μm, 10 μm and 5 μm in that order to remove the impurities. The resulting filtrate was mixed with aqueous 75% acetic acid solution to precipitate cellulose acetate propionate, and then washed with hot water at 70° C. until the wash waste could have a pH of from 6 to 7. Further, this was stirred in aqueous 0.001% calcium hydroxide solution for 0.5 hours, and then filtered. The resulting cellulose acetate propionate was dried at 70° C. Its ¹H-NMR confirmed that the thus-obtained cellulose acetate propionate has a degree of acetylation of 0.15, a degree of propionylation of 2.55, a weight-average molecular weight of 135000 and a number-average molecular weight of 52000.

The amount of acetic anhydride and that of propionic anhydride to be fed to the reaction system were varied to obtain cellulose acetate propionate having a degree of acetylation of 0.43, a degree of propionylation of 2.40, a weight-average molecular weight of 125000 and a number-average molecular weight of 48000.

This was pelletized under the same condition as in Example 1 of Example A to obtain pellets, of which the heat of crystalline fusion was 0.1 J/g, the number of acicular impurities was 0/mg, the sulfate group content was 70 ppm, M/S was 0.5, and M was Ca(OH)². Using the pellets, a film was formed in the same manner as in Example 1, for which, however, the touch roll contact pressure was 1 MPa. The thus-obtained film was tested, and its aged discoloration was 0.01, its yellowing in LCD was 0, or that is, the film had good properties.

### Example C

### (1) Production of Cellulose Acylate

0.1 parts by mass of acetic acid and 2.7 parts by mass of propionic acid were sprayed over 10 parts by mass of cellulose (hardwood pulp), and then stored at room temperature. The time for storage was changed whereby the amount of the acicular impurities was changed as in Table 3. Apart from this, a mixture of 1.2 parts by mass of acetic anhydride, 61 parts by mass of propionic anhydride and 0.7 parts by mass of sulfuric acid was prepared, cooled to −10° C., and mixed with the above pretreated cellulose in a reactor. After 30 minutes, the outer temperature was elevated up to 30° C., and the compounds were reacted for 4 hours (partial hydrolysis). 46 parts by mass of aqueous 25% acetic acid solution was added to the reactor, and the inner temperature was elevated up to 60° C., and this was stirred for 2 hours. 6.2 parts by mass of a solution prepared by mixing magnesium acetate 4-hydrate, acetic acid and water (1/1/1 by weight) was added to it, and stirred for 30 minutes. The reaction liquid was filtered under pressure through metal sintered filters having a retention particle size of 40 μm, 10 μm and 5 μm in that order to remove the impurities. The resulting filtrate was mixed with aqueous 75% acetic acid solution to precipitate cellulose acetate propionate, and then washed with hot water at 70° C. until the wash waste could have a pH of from 6 to 7. Further, this was stirred in aqueous 0.001% calcium hydroxide solution for 0.5 hours, and then filtered. The resulting cellulose acetate propionate was dried at 70° C. Its ¹H-NMR confirmed that the thus-obtained cellulose acetate propionate has a degree of acetylation of 0.15, a degree of propionylation of 2.55, a weight-average degree of polymerization of 420 and a number-average degree of polymerization of 160.

The amount of acetic anhydride and that of propionic anhydride to be fed to the reaction system were varied and butyric acid was added to the system to obtain cellulose acylate having a different degree of acetylation, propionylation and butyrylation as in Table 3. In addition, the time for partial hydrolysis was changed (when it is longer, then the degree of polymerization of the polymer is lower) to obtain cellulose acylate having a different weight-average degree of polymerization as in Table 3.

### (2) Formation of Cellulose Acylate Grains

The cellulose acylate shown in able 3 was dissolved in a solvent having an SP value as in Table 3 to prepare a cellulose acylate solution having a concentration of 10% by mass. This was processed according to any of the following methods (as in Table 3) give cellulose acylate grains.

(i) Precipitation Method:

A mixed solvent of acetic acid and water (acetic acid/water=1/1 by weight) was used as a poor solvent. The cellulose acylate solution was added to it, whereupon the number of revolution of the stirring blade was changed (when the number of stirring revolution is larger, then the grain size is smaller) to give cellulose acylate grains having the size as in Table 3. This was filtered, washed with water and dried.

(ii) Drying Method:

The cellulose acylate solution was put into a chamber, and dried at a temperature higher by 10° C. than the boiling point of the solvent so that the residual solvent was reduced to at most 0.01% by weight. Next, this was ground to give the cellulose acylate grains having the size as in Table 3. The size control of the grains was attained by controlling the grinding time and by further sieving the ground grains.

### (3) Melt-Casting Film Formation

The above cellulose acylate grains were dried in a drier at 110° C. (using air having a dew point of −20° C.) for 2 hours to reduce the residual water content to at most 0.01% by mass. These were put into the hopper controlled at (Tg−10)° C. of a double-screw extruder, and kneaded and melted therein in an air atmosphere. The first feed port temperature was 180° C.; the compression zone temperature was 220° C.; and the second feed port temperature was 230° C. The number of screw revolution was 100 rpm, and the kneading resin pressure was 5 MPa. At the outlet port of the extruder, the resin melt was filtered through a breaker plate-type filter, then led to pass through a gear pump, and again filtered through a 3-μm stainless leaf-type disc filter device.

The resulting resin melt was extruded out through the T-die of the extruder, and then formed into a film using the touch roll described in Example 1 in JP-A-11-235747 under a linear pressure shown in Table 3. The casting roll and the touch roll had a diameter of 500 mm. After led to pass through a series of 3 casting rolls set at 120° C. on the most upstream side, at 125° C. in the middle, and at 115° C. on the most downstream side, the film was trimmed at both edges (3% of the overall width at each edge). This was knurled to a width of 10 mm and a height of 30 μm at both edges, and then wound up at a speed of 30 m/min. Thus wound up, the film had a width of 1.5 m and a length of m.

TABLE 3

| | Cellulose Acylate | | | | | | |
|---|---|---|---|---|---|---|---|
| | Degree of Substitution | | | | | Weight | |
| | acetyl | propionyl | butyryl | Y | | Average | |
| | group | group | group | (total oF Y1 | | Degree of | Tg |
| | (X) | (Y1) | (Y2) | and Y2) | X + Y | Polymerization | (° C.) |
| Comparative Example 101 | 0.15 | 2.55 | | 2.55 | 2.70 | 420 | 137 |
| Example 101 | 0.15 | 2.55 | | 2.55 | 2.70 | 420 | 137 |
| Example 102 | 0.15 | 2.55 | | 2.55 | 2.70 | 420 | 137 |
| Example 103 | 0.15 | 2.55 | | 2.55 | 2.70 | 420 | 137 |
| Example 104 | 0.15 | 2.55 | | 2.55 | 2.70 | 420 | 137 |
| Example 105 | 0.15 | 2.55 | | 2.55 | 2.70 | 420 | 137 |
| Example 106 | 0.15 | 2.55 | | 2.55 | 2.70 | 420 | 137 |
| Comparative Example 102 | 0.15 | 2.55 | | 2.55 | 2.70 | 530 | 137 |
| Comparative Example 103 | 0.4 | 2.5 | | 2.5 | 2.9 | 400 | 137 |
| Example 107 | 0.4 | 2.5 | | 2.5 | 2.9 | 400 | 137 |
| Example 108 | 0.4 | 2.5 | | 2.5 | 2.9 | 400 | 137 |
| Example 109 | 0.4 | 2.5 | | 2.5 | 2.9 | 400 | 137 |
| Comparative Example 104 | 0.4 | 2.5 | | 2.5 | 2.9 | 400 | 137 |
| Example 110 | 0.4 | 2.5 | | 2.5 | 2.9 | 400 | 137 |
| Example 111 | 0.4 | 2.5 | | 2.5 | 2.9 | 400 | 137 |
| Example 112 | 0.4 | 2.5 | | 2.5 | 2.9 | 400 | 137 |
| Example 113 | 0.4 | 2.5 | | 2.5 | 2.9 | 400 | 137 |
| Example 114 | 0.4 | 2.5 | | 2.5 | 2.9 | 400 | 137 |
| Example 115 | 0.4 | 2.5 | | 2.5 | 2.9 | 400 | 137 |
| Example 116 | 0.4 | 2.5 | | 2.5 | 2.9 | 400 | 137 |
| Example 117 | 0.4 | 2.5 | | 2.5 | 2.9 | 400 | 137 |
| Example 118 | 0.4 | 2.5 | | 2.5 | 2.9 | 400 | 137 |
| Example 119 | 0.4 | 2.5 | | 2.5 | 2.9 | 400 | 137 |
| Example 120 | 0.4 | 2.5 | | 2.5 | 2.9 | 400 | 137 |
| Example 121 | 0.4 | 2.5 | | 2.5 | 2.9 | 400 | 137 |
| Example 122 | 0.4 | 2.5 | | 2.5 | 2.9 | 400 | 137 |
| Example 123 | 0.4 | 2.5 | | 2.5 | 2.9 | 400 | 137 |
| Example 124 | 0.4 | 2.5 | | 2.5 | 2.9 | 400 | 137 |
| Example 125 | 0.4 | 2.5 | | 2.5 | 2.9 | 220 | 135 |
| Example 126 | 0.4 | 2.5 | | 2.5 | 2.9 | 260 | 137 |
| Example 127 | 0.4 | 2.5 | | 2.5 | 2.9 | 480 | 137 |
| Example 128 | 0.4 | 2.5 | | 2.5 | 2.9 | 530 | 137 |
| Example 129 | 0.05 | 2.95 | | 2.95 | 3.0 | 450 | 138 |
| Example 130 | 0.8 | 2.0 | | 2.0 | 2.8 | 450 | 130 |
| Example 131 | 1.6 | 1.2 | | 1.2 | 2.8 | 450 | 140 |
| Example 132 | 2.0 | 0.8 | | 0.8 | 2.8 | 450 | 145 |
| Example 133 | 0.6 | 2.0 | | 2.0 | 2.6 | 450 | 132 |
| Example 134 | 0.4 | 2.0 | | 2.0 | 2.4 | 450 | 135 |
| Example 135 | 1.5 | | 1.2 | 1.2 | 2.7 | 450 | 135 |
| Example 136 | 1.0 | | 1.7 | 1.7 | 2.7 | 450 | 130 |
| Example 137 | 0.4 | | 2.5 | 2.5 | 2.9 | 450 | 125 |

| | | | Formation of | | Properties of Cellulose | |
|---|---|---|---|---|---|---|
| | | | Cellulose | | Acylate Grains | |
| | | | Acylate Grains | | Heat of | |
| | Dissolution of | | | Size of | Crystalline | Acicular |
| | Cellulose Acylate | | | Grains | Fusion | Impurities |
| | Type of Solvent | SP Value | Method | (mm³) | (J/g) | (/mg) |
| Comparative Example 101 | neopentane | 6.3 | precipitation method | 5 | 12 | 0 |
| Example 101 | diethyl ether | 7.4 | precipitation method | 5 | 3 | 0 |
| Example 102 | ethyl acetate | 8.0 | precipitation method | 5 | 1 | 0 |
| Example 103 | butyl acetate | 8.5 | precipitation method | 5 | 1 | 0 |
| Example 104 | methyl ethyl ketone | 9.3 | precipitation method | 5 | 1 | 0 |
| Example 105 | dichloromethane | 9.5 | precipitation method | 5 | 1 | 0 |
| Example 106 | acetone | 9.9 | precipitation method | 5 | 5 | 0 |
| Comparative Example 102 | acetic acid | 10.1 | precipitation method | 5 | 15 | 0 |
| Comparative Example 103 | neopentane | 6.3 | drying method | 100 | 12 | 0 |
| Example 107 | diethyl ether | 7.4 | drying method | 100 | 2 | 0 |
| Example 108 | dichloromethane | 9.5 | drying method | 100 | 1 | 0 |
| Example 109 | acetone | 9.9 | drying method | 100 | 3 | 0 |
| Comparative Example 104 | acetic acid | 10.1 | drying method | 100 | 14 | 0 |
| Example 110 | ethyl acetate | 8.0 | precipitation method | 40 | 1 | 60 |
| Example 111 | ethyl acetate | 8.0 | precipitation method | 40 | 1 | 45 |
| Example 112 | ethyl acetate | 8.0 | precipitation method | 40 | 1 | 20 |
| Example 113 | butyl acetate | 8.5 | drying method | 8000 | 1 | 0 |
| Example 114 | butyl acetate | 8.5 | drying method | 8000 | 1 | 0 |
| Example 115 | butyl acetate | 8.5 | drying method | 8000 | 1 | 0 |
| Example 116 | butyl acetate | 8.5 | drying method | 8000 | 1 | 0 |
| Example 117 | dichloromethane | 9.5 | precipitation method | 2 | 1 | 0 |
| Example 118 | dichloramethane | 9.5 | precipitation method | 2 | 1 | 0 |
| Example 119 | dichloramethane | 9.5 | precipitation method | 2 | 1 | 0 |
| Example 120 | dichloromethane | 9.5 | precipitation method | 2 | 1 | 0 |
| Example 121 | acetone | 9.9 | drying method | 1000 | 5 | 0 |
| Example 122 | acetone | 9.9 | drying method | 1000 | 5 | 0 |
| Example 123 | acetone | 9.9 | drying method | 1000 | 5 | 0 |
| Example 124 | acetone | 9.9 | drying method | 1000 | 5 | 0 |
| Example 125 | ethyl acetate | 8.0 | precipitation method | 80 | 3 | 0 |
| Example 126 | ethyl acetate | 8.0 | precipitation method | 80 | 1 | 0 |
| Example 127 | ethyl acetate | 8.0 | precipitation method | 80 | 1 | 0 |
| Example 128 | ethyl acetate | 8.0 | precipitation method | 80 | 3 | 0 |
| Example 129 | dichloromethane | 9.5 | precipitation method | 50 | 2 | 0 |
| Example 130 | dichloromethane | 9.5 | precipitation method | 50 | 1 | 0 |
| Example 131 | dichloromethane | 9.5 | precipitation method | 50 | 2 | 0 |
| Example 132 | dichloromethane | 9.5 | precipitation method | 50 | 6 | 0 |
| Example 133 | dichloromethane | 9.5 | precipitation method | 50 | 2 | 0 |
| Example 134 | dichloromethane | 9.5 | precipitation method | 50 | 4 | 0 |
| Example 135 | dichloromethane | 9.5 | precipitation method | 50 | 4 | 0 |
| Example 136 | dichloromethane | 9.5 | precipitation method | 50 | 2 | 0 |
| Example 137 | dichloromethane | 9.5 | precipitation method | 50 | 4 | 0 |

| | Properties of Cellulose | | | Film Formation | | Evaluation of Film | |
|---|---|---|---|---|---|---|---|
| | Acylate Grains | | | Touch Roll | | Aged | |
| | Sulfate | | | Contact | | Discoloration | |
| | group | | | Pressure | Thickness | (400 nm | Yellowing |
| | (ppm) | M/S* | Type of M* | (MPa) | (μm) | absorbance) | in LCD |
| Comparative Example 101 | 80 | 0.6 | Ca(OH)₂ | 1.0 | 80 | 0.39 | 9 |
| Example 101 | 80 | 0.6 | Ca(OH)₂ | 1.0 | 80 | 0.15 | 4 |
| Example 102 | 80 | 0.6 | Ca(OH)₂ | 1.0 | 80 | 0.08 | 1 |
| Example 103 | 80 | 0.6 | Ca(OH)₂ | 1.0 | 80 | 0.07 | 1 |
| Example 104 | 80 | 0.6 | Ca(OH)₂ | 1.0 | 80 | 0.08 | 1 |
| Example 105 | 80 | 0.6 | Ca(OH)₂ | 1.0 | 80 | 0.08 | 1 |
| Example 106 | 80 | 0.6 | Ca(OH)₂ | 1.0 | 80 | 0.18 | 4 |
| Comparative Example 102 | 80 | 0.6 | Ca(OH)₂ | 1.0 | 80 | 0.45 | 9 |
| Comparative Example 103 | 40 | 0.8 | Mg(OH)₂ | 1.6 | 100 | 0.38 | 9 |
| Example 107 | 40 | 0.8 | Mg(OH)₂ | 1.6 | 100 | 0.14 | 4 |
| Example 108 | 40 | 0.8 | Mg(OH)₂ | 1.6 | 100 | 0.08 | 1 |
| Example 109 | 40 | 0.8 | Mg(OH)₂ | 1.6 | 100 | 0.16 | 4 |
| Comparative Example 104 | 40 | 0.8 | Mg(OH)₂ | 1.6 | 100 | 0.49 | 9 |
| Example 110 | 120 | 1.2 | Mg(OH)₂ | 1.4 | 280 | 0.21 | 4 |
| Example 111 | 120 | 1.2 | Mg(OH)₂ | 1.4 | 280 | 0.13 | 1 |
| Example 112 | 120 | 1.2 | Mg(OH)₂ | 1.4 | 280 | 0.08 | 1 |
| Example 113 | 220 | 1.2 | Ca(OH)₂ | 0.8 | 40 | 0.16 | 5 |
| Example 114 | 180 | 1.2 | Ca(OH)₂ | 0.8 | 40 | 0.10 | 2 |
| Example 115 | 30 | 1.2 | Ca(OH)₂ | 0.8 | 40 | 0.07 | 1 |
| Example 116 | 0 | 1.2 | Ca(OH)₂ | 0.8 | 40 | 0.07 | 0 |
| Example 117 | 100 | 3.2 | Ca(OH)₂ | 1.2 | 60 | 0.15 | 5 |
| Example 118 | 100 | 2.8 | Ca(OH)₂ | 1.2 | 60 | 0.08 | 2 |
| Example 119 | 100 | 0.4 | Ca(OH)₂ | 1.2 | 60 | 0.05 | 1 |
| Example 120 | 100 | 0.2 | Ca(OH)₂ | 1.2 | 60 | 0.16 | 5 |
| Example 121 | 60 | 1.5 | NaH(CO₃) | no used | 80 | 0.19 | 5 |
| Example 122 | 60 | 1.5 | NaH(CO₃) | 0.3 | 80 | 0.12 | 3 |
| Example 123 | 60 | 1.5 | NaH(CO₃) | 3.0 | 80 | 0.05 | 1 |
| Example 124 | 60 | 1.5 | NaH(CO₃) | 4.0 | 80 | 0.13 | 3 |
| Example 125 | 40 | 0.6 | KH(CO₃) | 1.8 | 120 | 0.15 | 4 |
| Example 126 | 40 | 0.6 | KH(CO₃) | 1.8 | 120 | 0.08 | 1 |
| Example 127 | 40 | 0.6 | KH(CO₃) | 1.8 | 120 | 0.07 | 2 |
| Example 128 | 40 | 0.6 | KH(CO₃) | 1.8 | 120 | 0.18 | 5 |
| Example 129 | 150 | 2.2 | Ca(OH)₂ | 2.2 | 150 | 0.11 | 1 |
| Example 130 | 150 | 2.2 | Ca(OH)₂ | 2.2 | 150 | 0.12 | 1 |
| Example 131 | 150 | 2.2 | Ca(OH)₂ | 2.2 | 150 | 0.10 | 2 |
| Example 132 | 150 | 2.2 | Ca(OH)₂ | 2.2 | 150 | 0.18 | 5 |
| Example 133 | 150 | 2.2 | Ca(OH)₂ | 2.2 | 150 | 0.07 | 2 |
| Example 134 | 150 | 2.2 | Ca(OH)₂ | 2.2 | 150 | 0.18 | 4 |
| Example 135 | 150 | 2.2 | Ca(OH)₂ | 2.2 | 150 | 0.09 | 1 |
| Example 136 | 150 | 2.2 | Ca(OH)₂ | 2.2 | 150 | 0.10 | 1 |
| Example 137 | 150 | 2.2 | Ca(OH)₂ | 2.2 | 150 | 0.11 | 1 |

*M: sum of molar amount of alkali metal and molar amount of alkaline earth metal.
S: molar amount of sulfate group.

### (4) Stretching

Stretched cellulose acylate films were obtained in the same manner as in Example A. Like in Example A, the films of the invention of these Examples had good properties.

### (5) Test of Unstretched or Stretched Cellulose Acylate Film for High-Temperature Aged Discoloration

The films were tested and evaluated in the same manner as in Example A (aged discoloration), and the results are shown in Table 3.

### (6) Construction of Polarizer

In the same manner as in Example A, the films were saponified by dipping, a polarizing film was prepared, and they were laminated and built in liquid-crystal display devices, and tested. The results are shown in Table 3 (yellowing in LCD). The films of the invention of these Examples all had good optical properties.

### (7) Optical Compensatory Film

In the same manner as in Example A, the unstretched or stretched cellulose acylate film of the invention was used in forming optical compensatory films, and they had good optical properties.

### (8) Low-Refractivity Film

In the same manner as in Example A, the cellulose acylate film of the invention was used in forming low-refractivity films, and they had good optical properties.

### (9) Construction of Liquid-Crystal Display Device

In the same manner as in Example A, liquid-crystal display devices were constructed, and those comprising the cellulose acylate film of the invention had good optical properties.

The cellulose acylate grains of Example 101, and the cellulose acylate pellets of Example 1 were mixed (10/90, 30/70, 70/30, 10/90), and the resulting mixture was formed into a film and stretched in the same manner as in Example 101, and used in forming polarizers, optical compensatory films, low-refractivity films, and liquid-crystal display devices. These also produced good results.

### INDUSTRIAL APPLICABILITY

The cellulose acylate film of the invention does not yellow, when built in liquid-crystal display devices and used for a long period of time. Accordingly, the cellulose acylate film of the invention is extremely useful as polarizers, optical compensatory films, and antireflection films. In addition, according to the cellulose acylate grains and the method for their production of the invention, the cellulose acylate film can be produced in a simplified manner. Accordingly, the industrial applicability of the invention is great.

1. Cellulose acylate grains having a heat quantity of crystalline fusion of at most 10 J/g.

2. The cellulose acylate grains according to claim 1, wherein the number of acicular impurities is at most 50/mg.

3. The cellulose acylate grains according to claim 1, having a sulfate group content of from 0 ppm to less than 200 ppm.

4. The cellulose acylate grains according to claim 1, wherein the ratio of (sum of the molar amount of alkali metal and the molar amount of Group-2 metal)/(the molar amount of sulfate group) is from 0.3 to 3.0.

5. The cellulose acylate grains according to claim 4, wherein the Group-2 metal is calcium.

6. The cellulose acylate grains according to claim 1, satisfying the following formulae (S-1) to (S-3):
2.6≦X+Y≦3.0, (S-1)
0≦X≦1.8, (S-2)
1.0≦Y≦3; (S-3)
wherein X means a degree of substitution of the hydroxyl group of cellulose for an acetyl group; Y means a total degree of substitution of the hydroxyl group of cellulose for a propionyl group, a butyryl group, a pentanoyl group and a hexanoyl group.

7. The cellulose acylate grains according to claim 1, which are pellets.

8. A method for producing cellulose acylate grains, which comprises kneading a cellulose acylate resin in a double-screw kneading extruder at a screw revolution of from 50 to 300 rpm and under a resin-kneading pressure of 2 to 9 MPa.

9. The method for producing cellulose acylate grains according to claim 8, which includes kneading and extruding the cellulose acylate resin at 160° C. to 220° C. and pelletizing it.

10. The method for producing cellulose acylate grains according to claim 8, wherein the resin is pelletized by controlling the inner pressure of the double-screw kneading extruder to lower than 1 atmospheric pressure.

11. The method for producing cellulose acylate grains according to claim 8, wherein the resin is pelletized while an inert gas is introduced into the double-screw kneading extruder.

12. The method for producing cellulose acylate grains according to claim 9, which includes grinding the pellets formed through pelletization.

13. The method for producing cellulose acylate grains according to claim 8, which includes reacting the cellulose acylate with a carbonate, hydrogencarbonate, hydroxide or oxide of at least one metal selected from the group consisting of sodium, potassium, magnesium and calcium for neutralization before the kneading.

14. A method for producing cellulose acylate grains, which comprises preparing a cellulose acylate solution by dissolving a cellulose acylate in a solvent having an SP value of from 7 to 10, and then solidifying the cellulose acylate.

15. The method for producing cellulose acylate grains according to claim 14, wherein the solvent having an SP value of from 7 to 10 is an ester solvent having an SP value of from 7 to 10, a halogenated hydrocarbon solvent having an SP value of from 7 to 10 or a ketone solvent having an SP value of from 7 to 10.

16. The method for producing cellulose acylate grains according to claim 14, wherein the solidification is attained by drying the cellulose acylate solution to remove the solvent.

17. The method for producing cellulose acylate grains according to claim 14, wherein the solidification is attained by introducing the cellulose acylate solution into a poor solvent to thereby precipitate the cellulose acylate.

18. A method for producing a cellulose acylate film, which comprises melt-casting cellulose acylate grains of claim 1 into a film.

19. The method for producing a cellulose acylate film according to claim 18, wherein the film is formed by the use of a touch roll under a linear pressure of from 3 kg/cm to 100 kg/cm.

20. The method for producing a cellulose acylate film according to claim 18, wherein the film is formed by the use of a touch roll under a contact pressure of from 0.3 MPa to 3 MPa.

21. The method for producing a cellulose acylate film according to claim 18, which further includes stretching the formed cellulose acylate film in at least one direction by from 1% to 300%.

21. The method for producing a cellulose acylate film according to claim 18, which further includes stretching the formed cellulose acylate film in at least one direction by from 1% to 300%.

22. A cellulose acylate film produced according to the production method of claim 18.

23. A cellulose acylate film formed of the cellulose acylate grains of claim 1, which has a residual solvent content of at most 0.01% by mass.

24. A polarizer comprising a polarizing film and at least one layer of the cellulose acylate film of claim 22 laminated thereon.

25. An optical compensatory film comprising the cellulose acylate film of claim 22 as the substrate thereof.

26. An antireflection film comprising the cellulose acylate film of claim 22 as the substrate thereof.

27. A liquid-crystal display device comprising the cellulose acylate film of claim 22.
